SEQUENCE LISTING (18 sequences)

SEQ ID NO: 1 - protein, 15 aa, Artificial Sequence
  Chemically synthesized peptide
KGGPQVTRGDVFTMP

SEQ ID NO: 2 - protein, 15 aa, Artificial Sequence
  Chemically synthesized peptide
KYGAASIKVAVSADR

SEQ ID NO: 3 - protein, 7 aa, Artificial Sequence
  Chemically synthesized peptide
GRGDSPK

SEQ ID NO: 4 - protein, 15 aa, Artificial Sequence
  Chemically synthesized peptide
KGGAVTGRGDSPASS

SEQ ID NO: 5 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 6 - protein, 2355 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 7 - protein, 3075 aa, Homo sapiens
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 8 - protein, 3122 aa, Homo sapiens
MPGAAGVLLLLLLSGGLGGVQAQRPQQQRQSQAHQQRGLFPAVLNLASNALITTNATCGEKGPEMYCKLVEHVPGQPVRNPQCRICNQNSSNPNQRHPITNAIDGKNTWWQSPSIKNGIEYHYVTITLDLQQVFQIAYVIVKAANSPRPGNWILERSLDDVEYKPWQYHAVTDTECLTLYNIYPRTGPPSYAKDDEVICTSFYSKIHPLENGEIHISLINGRPSADDPSPELLEFTSARYIRLRFQRIRTLNADLMMFAHKDPREIDPIVTRRYYYSVKDISVGGMCICYGHARACPLDPATNKSRCECEHNTCGDSCDQCCPGFHQKPWRAGTFLTKTECEACNCHGKAEECYYDENVARRNLSLNIRGKYIGGGVCINCTQNTAGINCETCTDGFFRPKGVSPNYPRPCQPCHCDPIGSLNEVCVKDEKHARRGLAPGSCHCKTGFGGVSCDRCARGYTGYPDCKACNCSGLGSKNEDPCFGPCICKENVEGGDCSRCKSGFFNLQEDNWKGCDECFCSGVSNRCQSSYWTYGKIQDMSGWYLTDLPGRIRVAPQQDDLDSPQQISISNAEARQALPHSYYWSAPAPYLGNKLPAVGGQLTFTISYDLEEEEEDTERVLQLMIILEGNDLSISTAQDEVYLHPSEEHTNVLLLKEESFTIHGTHFPVRRKEFMTVLANLKRVLLQITYSFGMDAIFRLSSVNLESAVSYPTDGSIAAAVEVCQCPPGYTGSSCESCWPRHRRVNGTIFGGICEPCQCFGHAESCDDVTGECLNCKDHTGGPYCDKCLPGFYGEPTKGTSEDCQPCACPLNIPSNNFSPTCHLDRSLGLICDGCPVGYTGPRCERCAEGYFGQPSVPGGSCQPCQCNDNLDFSIPGSCDSLSGSCLICKPGTTGRYCELCADGYFGDAVDAKNCQPCRCNAGGSFSEVCHSQTGQCECRANVQGQRCDKCKAGTFGLQSARGCVPCNCNSFGSKSFDCEESGQCWCQPGVTGKKCDRCAHGYFNFQEGGCTACECSHLGNNCDPKTGRCICPPNTIGEKCSKCAPNTWGHSITTGCKACNCSTVGSLDFQCNVNTGQCNCHPKFSGAKCTECSRGHWNYPRCNLCDCFLPGTDATTCDSETKKCSCSDQTGQCTCKVNVEGIHCDRCRPGKFGLDAKNPLGCSSCYCFGTTTQCSEAKGLIRTWVTLKAEQTILPLVDEALQHTTTKGIVFQHPEIVAHMDLMREDLHLEPFYWKLPEQFEGKKLMAYGGKLKYAIYFEAREETGFSTYNPQVIIRGGTPTHARIIVRHMAAPLIGQLTRHEIEMTEKEWKYYGDDPRVHRTVTREDFLDILYDIHYILIKATYGNFMRQSRISEISMEVAEQGRGTTMTPPADLIEKCDCPLGYSGLSCEACLPGFYRLRSQPGGRTPGPTLGTCVPCQCNGHSSLCDPETSICQNCQHHTAGDFCERCALGYYGIVKGLPNDCQQCACPLISSSNNFSPSCVAEGLDDYRCTACPRGYEGQYCERCAPGYTGSPGNPGGSCQECECDPYGSLPVPCDPVTGFCTCRPGATGRKCDGCKHWHAREGWECVFCGDECTGLLLGDLARLEQMVMSINLTGPLPAPYKMLYGLENMTQELKHLLSPQRAPERLIQLAEGNLNTLVTEMNELLTRATKVTADGEQTGQDAERTNTRAKSLGEFIKELARDAEAVNEKAIKLNETLGTRDEAFERNLEGLQKEIDQMIKELRRKNLETQKEIAEDELVAAEALLKKVKKLFGESRGENEEMEKDLREKLADYKNKVDDAWDLLREATDKIREANRLFAVNQKNMTALEKKKEAVESGKRQIENTLKEGNDILDEANRLADEINSIIDYVEDIQTKLPPMSEELNDKIDDLSQEIKDRKLAEKVSQAESHAAQLNDSSAVLDGILDEAKNISFNATAAFKAYSNIKDYIDEAEKVAKEAKDLAHEATKLATGPRGLLKEDAKGCLQKSFRILNEAKKLANDVKENEDHLNGLKTRIENADARNGDLLRTLNDTLGKLSAIPNDTAAKLQAVKDKARQANDTAKDVLAQITELHQNLDGLKKNYNKLADSVAKTNAVVKDPSKNKIIADADATVKNLEQEADRLIDKLKPIKELEDNLKKNISEIKELINQARKQANSIKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLSMKARPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTGKPLEVNFAKALELRGVQPVSCPAN

SEQ ID NO: 9 - protein, 1724 aa, Homo sapiens
MPPAVRRSACSMGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGSPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 10 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 11 - protein, 3695 aa, Homo sapiens
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTTGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSATCANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPAMSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPKGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVFLRRVALEVASPAGQGALASNVELCLCPASYRGDSCQECAPGFYRDVKGLFLGRCVPCQCHGHSDRCLPGSGVCVDCQHNTEGAHCERCQAGFVSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFDGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAVGTRDQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 12 - protein, 1786 aa, Homo sapiens
MGLLQLLAFSFLALCRARVRAQEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGNCFCYGHASECAPVDGFNEEVEGMVHGHCMCRHNTKGLNCELCMDFYHDLPWRPAEGRNSNACKKCNCNEHSISCHFDMAVYLATGNVSGGVCDDCQHNTMGRNCEQCKPFYYQHPERDIRDPNFCERCTCDPAGSQNEGICDSYTDFSTGLIAGQCRCKLNVEGEHCDVCKEGFYDLSSEDPFGCKSCACNPLGTIPGGNPCDSETGHCYCKRLVTGQHCDQCLPEHWGLSNDLDGCRPCDCDLGGALNNSCFAESGQCSCRPHMIGRQCNEVEPGYYFATLDHYLYEAEEANLGPGVSIVERQYIQDRIPSWTGAGFVRVPEGAYLEFFIDNIPYSMEYDILIRYEPQLPDHWEKAVITVQRPGRIPTSSRCGNTIPDDDNQVVSLSPGSRYVVLPRPVCFEKGTNYTVRLELPQYTSSDSDVESPYTLIDSLVLMPYCKSLDIFTVGGSGDGVVTNSAWETFQRYRCLENSRSVVKTPMTDVCRNIIFSISALLHQTGLACECDPQGSLSSVCDPNGGQCQCRPNVVGRTCNRCAPGTFGFGPSGCKPCECHLQGSVNAFCNPVTGQCHCFQGVYARQCDRCLPGHWGFPSCQPCQCNGHADDCDPVTGECLNCQDYTMGHNCERCLAGYYGDPIIGSGDHCRPCPCPDGPDSGRQFARSCYQDPVTLQLACVCDPGYIGSRCDDCASGYFGNPSEVGGSCQPCQCHNNIDTTDPEACDKETGRCLKCLYHTEGEHCQFCRFGYYGDALQQDCRKCVCNYLGTVQEHCNGSDCQCDKATGQCLCLPNVIGQNCDRCAPNTWQLASGTGCDPCNCNAAHSFGPSCNEFTGQCQCMPGFGGRTCSECQELFWGDPDVECRACDCDPRGIETPQCDQSTGQCVCVEGVEGPRCDKCTRGYSGVFPDCTPCHQCFALWDVIIAELTNRTHRFLEKAKALKISGVIGPYRETVDSVERKVSEIKDILAQSPAAEPLKNIGNLFEEAEKLIKDVTEMMAQVEVKLSDTTSQSNSTAKELDSLQTEAESLDNTVKELAEQLEFIKNSDIRGALDSITKYFQMSLEAEERVNASTTEPNSTVEQSALMRDRVEDVMMERESQFKEKQEEQARLLDELAGKLQSLDLSAAAEMTCGTPPGASCSETECGGPNCRTDEGERKCGGPGCGGLVTVAHNAWQKAMDLDQDVLSALAEVEQLSKMVSEAKLRADEAKQSAEDILLKTNATKEKMDKSNEELRNLIKQIRNFLTQDSADLDSIEAVANEVLKMEMPSTPQQLQNLTEDIRERVESLSQVEVILQHSAADIARAEMLLEEAKRASKSATDVKVTADMVKEALEEAEKAQVAAEKAIKQADEDIQGTQNLLTSIESETAASEETLFNASQRISELERNVEELKRKAAQNSGEAEYIEKVVYTVKQSAEDVKKTLDGELDEKYKKVENLIAKKTEESADARRKAEMLQNEAKTLLAQANSKLQLLKDLERKYEDNQRYLEDKAQELARLEGEVRSLLKDISQKVAVYSTCL

SEQ ID NO: 13 - protein, 1798 aa, Homo sapiens
MELTSRERGRGQPLPWELRLGLLLSVLAATLAQAPAPDVPGCSRGSCYPATGDLLVGRADRLTASSTCGLNGPQPYCIVSHLQDEKKCFLCDSRRPFSARDNPHSHRIQNVVTSFAPQRRAAWWQSENGIPAVTIQLDLEAEFHFTHLIMTFKTFRPAAMLVERSADFGRTWHVYRYFSYDCGADFPGVPLAPPRHWDDVVCESRYSEIEPSTEGEVIYRVLDPAIPIPDPYSSRIQNLLKITNLRVNLTRLHTLGDNLLDPRREIREKYYYALYELVVRGNCFCYGHASECAPAPGAPAHAEGMVHGACICKHNTRGLNCEQCQDFYRDLPWRPAEDGHSHACRKCECHGHTHSCHFDMAVYLASGNVSGGVCDGCQHNTAGRHCELCRPFFYRDPTKDLRDPAVCRSCDCDPMGSQDGGRCDSHDDPALGLVSGQCRCKEHVVGTRCQQCRDGFFGLSISDRLGCRRCQCNARGTVPGSTPCDPNSGSCYCKRLVTGRGCDRCLPGHWGLSHDLLGCRPCDCDVGGALDPQCDEGTGQCHCRQHMVGRRCEQVQPGYFRPFLDHLIWEAEDTRGQVLDVVERLVTPGETPSWTGSGFVRLQEGQTLEFLVASVPKAMDYDLLLRLEPQVPEQWAELELIVQRPGPVPAHSLCGHLVPKDDRIQGTLQPHARYLIFPNPVCLEPGISYKLHLKLVRTGGSAQPETPYSGPGLLIDSLVLLPRVLVLEMFSGGDAAALERQATFERYQCHEEGLVPSKTSPSEACAPLLISLSTLIYNGALPCQCNPQGSLSSECNPHGGQCLCKPGVVGRRCDLCAPGYYGFGPTGCQACQCSHEGALSSLCEKTSGQCLCRTGAFGLRCDRCQRGQWGFPSCRPCVCNGHADECNTHTGACLGCRDHTGGEHCERCIAGFHGDPRLPYGGQCRPCPCPEGPGSQRHFATSCHQDEYSQQIVCHCRAGYTGLRCEACAPGHFGDPSRPGGRCQLCECSGNIDPMDPDACDPHTGQCLRCLHHTEGPHCAHCKPGFHGQAARQSCHRCTCNLLGTNPQQCPSPDQCHCDPSSGQCPCLPNVQGPSCDRCAPNFWNLTSGHGCQPCACHPSRARGPTCNEFTGQCHCRAGFGGRTCSECQELHWGDPGLQCHACDCDSRGIDTPQCHRFTGHCSCRPGVSGVRCDQCARGFSGIFPACHPCHACFGDWDRVVQDLAARTQRLEQRAQELQQTGVLGAFESSFWHMQEKLGIVQGIVGARNTSAASTAQLVEATEELRREIGEATEHLTQLEADLTDVQDENFNANHALSGLERDRLALNLTLRQLDQHLDLLKHSNFLGAYDSIRHAHSQSAEAERRANTSALAVPSPVSNSASARHRTEALMDAQKEDFNSKHMANQRALGKLSAHTHTLSLTDINELVCGAPGDAPCATSPCGGAGCRDEDGQPRCGGLSCNGAAATADLALGRARHTQAELQRALAEGGSILSRVAETRRQASEAQQRAQAALDKANASRGQVEQANQELQELIQSVKDFLNQEGADPDSIEMVATRVLELSIPASAEQIQHLAGAIAERVRSLADVDAILARTVGDVRRAEQLLQDARRARSWAEDEKQKAETVQAALEEAQRAQGIAQGAIRGAVADTRDTEQTLYQVQERMAGAERALSSAGERARQLDALLEALKLKRAGNSLAASTAEETAGSAQGRAQEAEQLLRGPLGDQYQTVKALAERKAQGVLAAQARAEQLRDEARDLLQAAQDKLQRLQELEGTYEENERALESKAAQLDGLEARMRSVLQAINLQVQIYNTCQ

SEQ ID NO: 14 - protein, 1172 aa, Homo sapiens [sequence omitted]

SEQ ID NO: 15 - protein, 1761 aa, Homo sapiens
MQFQLTLFLHLGWLSYSKAQDDCNRGACHPTTGDLLVGRNTQLMASSTCGLSRAQKYCILSYLEGEQKCFICDSRFPYDPYDQPNSHTIENVIVSFEPDREKKWWQSENGLDHVSIRLDLEALFRFSHLILTFKTFRPAAMLVERSTDYGHNWKVFKYFAKDCATSFPNITSGQAQGVGDIVCDSKYSDIEPSTGGEVVLKVLDPSFEIENPYSPYIQDLVTLTNLRINFTKLHTLGDALLGRRQNDSLDKYYYALYEMIVRGSCFCNGHASECRPMQKMRGDVFSPPGMVHGQCVCQHNTDGPNCERCKDFFQDAPWRPAADLQDNACRSCSCNSHSSRCHFDMTTYLASGGLSGGVCEDCQHNTEGQHCDRCRPLFYRDPLKTISDPYACIPCECDPDGTISGGICVSHSDPALGSVAGQCLCKENVEGAKCDQCKPNHYGLSATDPLGCQPCDCNPLGSLPFLTCDVDTGQCLCLSYVTGAHCEECTVGYWGLGNHLHGCSPCDCDIGGAYSNVCSPKNGQCECRPHVTGRSCSEPAPGYFFAPLNFYLYEAEEATTLQGLAPLGSETFGQSPAVHVVLGEPVPGNPVTWTGPGFARVLPGAGLRFAVNNIPFPVDFTIAIHYETQSAADWTVQIVVNPPGGSEHCIPKTLQSKPQSFALPAATRIMLLPTPICLEPDVQYSIDVYFSQPLQGESHAHSHVLVDSLGLIPQINSLENFCSKQDLDEYQLHNCVEIASAMGPQVLPGACERLIISMSAKLHDGAVACKCHPQGSVGSSCSRLGGQCQCKPLVVGRCCDRCSTGSYDLGHHGCHPCHCHPQGSKDTVCDQVTGQCPCHGEVSGRRCDRCLAGYFGFPSCHPCPCNRFAELCDPETGSCFNCGGFTTGRNCERCIDGYYGNPSSGQPCRPCLCPDDPSSNQYFAHSCYQNLWSSDVICNCLQGYTGTQCGECSTGFYGNPRISGAPCQPCACNNNIDVTDPESCSRVTGECLRCLHNTQGANCQLCKPGHYGSALNQTCRRCSCHASGVSPMECPPGGGACLCDPVTGACPCLPNVTGLACDRCADGYWNLVPGRGCQSCDCDPRTSQSSHCDQLTGQCPCKLGYGGKRCSECQENYYGDPPGRCIPCDCNRAGTQKPICDPDTGMCRCREGVSGQRCDRCARGHSQEFPTCLQCHLCFDQWDHTISSLSKAVQGLMRLAANMEDKRETLPVCEADFKDLRGNVSEIERILKHPVFPSGKFLKVKDYHDSVRRQIMQLNEQLKAVYEFQDLKDTIERAKNEADLLLEDLQEEIDLQSSVLNASIADSSENIKKYYHISSSAEKKINETSSTINTSANTRNDLLTILDTLTSKGNLSLERLKQIKIPDIQILNEKVCGDPGNVPCVPLPCGGALCTGRKGHRKCRGPGCHGSLTLSTNALQKAQEAKSIIRNLDKQVRGLKNQIESISEQAEVSKNNALQLREKLGNIRNQSDSEEENINLFIKKVKNFLLEENVPPEDIEKVANGVLDIHLPIPSQNLTDELVKIQKHMQLCEDYRTDENRLNEEADGAQKLLVKAKAAEKAANILLNLDKTLNQLQQAQITQGRANSTITQLTANITKIKKNVLQAENQTREMKSELELAKQRSGLEDGLSLLQTKLQRHQDHAVNAKVQAESAQHQAGSLEKEFVELKKQYAILQRKTSTTGLTKETLGKVKQLKDAAEKLAGDTEAKIRRITDLERKIQDLNLSRQAKADQLRILEDQVVAIKNEIVEQEKKYARCYS

SEQ ID NO: 16 - protein, 1609 aa, Homo sapiens
MRGSHRAAPALRPRGRLWPVLAVLAAAAAAGCAQAAMDECTDEGGRPQRCMPEFVNAAFNVTVVATNTCGTPPEEYCVQTGVTGVTKSCHLCDAGQPHLQHGAAFLTDYNNQADTTWWQSQTMLAGVQYPSSINLTLHLGKAFDITYVRLKFHTSRPESFAIYKRTREDGPWIPYQYYSGSCENTYSKANRGFIRTGGDEQQALCTDEFSDISPLTGGNVAFSTLEGRPSAYNFDNSPVLQEWVTATDIRVTLNRLNTFGDEVFNDPKVLKSYYYAISDFAVGGRCKCNGHASECMKNEFDKLVCNCKHNTYGVDCEKCLPFFNDRPWRRATAESASECLPCDCNGRSQECYFDPELYRSTGHGGHCTNCQDNTDGAHCERCRENFFRLGNNEACSSCHCSPVGSLSTQCDSYGRCSCKPGVMGDKCDRCQPGFHSLTEAGCRPCSCDPSGSIDECNIETGRCVCKDNVEGFNCERCKPGFFNLESSNPRGCTPCFCFGHSSVCTNAVGYSVYSISSTFQIDEDGWRAEQRDGSEASLEWSSERQDIAVISDSYFPRYFIAPAKFLGKQVLSYGQNLSFSFRVDRRDTRLSAEDLVLEGAGLRVSVPLIAQGNSYPSETTVKYVFRLHEATDYPWRPALTPFEFQKLLNNLTSIKIRGTYSERSAGYLDDVTLASARPGPGVPATWVESCTCPVGYGGQFCEMCLSGYRRETPNLGPYSPCVLCACNGHSETCDPETGVCNCRDNTAGPHCEKCSDGYYGDSTAGTSSDCQPCPCPGGSSCAVVPKTKEVVCTNCPTGTTGKRCELCDDGYFGDPLGRNGPVRLCRLCQCSDNIDPNAVGNCNRLTGECLKCIYNTAGFYCDRCKDGFFGNPLAPNPADKCKACNCNLYGTMKQQSSCNPVTGQCECLPHVTGQDCGACDPGFYNLQSGQGCERCDCHALGSTNGQCDIRTGQCECQPGITGQHCERCEVNHFGFGPEGCKPCDCHPEGSLSLQCKDDGRCECREGFVGNRCDQCEENYFYNRSWPGCQECPACYRLVKDKVADHRVKLQELESLIANLGTGDEMVTDQAFEDRLKEAEREVMDLLREAQDVKDVDQNLMDRLQRVNNTLSSQISRLQNIRNTIEETGNLAEQARAHVENTERLIEIASRELEKAKVAAANVSVTQPESTGDPNNMTLLAEEARKLAERHKQEADDIVRVAKTANDTSTEAYNLLLRTLAGENQTAFEIEELNRKYEQAKNISQDLEKQAARVHEEAKRAGDKAVEIYASVAQLSPLDSETLENEANNIKMEAENLEQLIDQKLKDYEDLREDMRGKELEVKNLLEKGKTEQQTADQLLARADAAKALAEEAAKKGRDTLQEANDILNNLKDFDRRVNDNKTAAEEALRKIPAINQTITEANEKTREAQQALGSAAADATEAKNKAHEAERIASAVQKNATSTKAEAERTFAEVTDLDNEVNNMLKQLQEAEKELKRKQDDADQDMMMAGMASQAAQEAEINARKAKNSVTSLLSIINDLLEQLGQLDTVDLNKLNEIEGTLNKAKDEMKVSDLDRKVSDLENEAKKQEAAIMDYNRDIEEIMKDIRNLEDIRKTLPSGCFNTPSIEKP

SEQ ID NO: 17 - protein, 1193 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMDQPLSVDEEGLVLLEQKLSRAKTQINSQLRPMMSELEERARQQRGHLHLLETSIDGILADVKNLENIRDNLPPGCYNTQALEQQ

SEQ ID NO: 18 - protein, 1575 aa, Homo sapiens
MAAAALLLGLALLAPRAAGAGMGACYDGAGRPQRCLPVFENAAFGRLAQASHTCGSPPEDFCPHVGAAGAGAHCQRCDAADPQRHHNASYLTDFHSQDESTWWQSPSMAFGVQYPTSVNITLRLGKAYEITYVRLKFHTSRPESFAIYKRSRADGPWEPYQFYSASCQKTYGRPEGQYLRPGEDERVAFCTSEFSDISPLSGGNVAFSTLEGRPSAYNFEESPGLQEWVTSTELLISLDRLNTFGDDIFKDPKVLQSYYYAVSDFSVGGRCKCNGHASECGPDVAGQLACRCQHNTTGTDCERCLPFFQDRPWARGTAEAAHECLPCNCSGRSEECTFDRELFRSTGHGGRCHHCRDHTAGPHCERCQENFYHWDPRMPCQPCDCQSAGSLHLQCDDTGTCACKPTVTGWKCDRCLPGFHSLSEGGCRPCTCNPAGSLDTCDPRSGRCPCKENVEGNLCDRCRPGTFNLQPHNPAGCSSCFCYGHSKVCASTAQFQVHHILSDFHQGAEGWWARSVGGSEHPPQWSPNGVLLSPEDEEELTAPEKFLGDQRFSYGQPLILTFRVPPGDSPLPVQLRLEGTGLALSLRHSSLSGPQDAGHPREVELRFHLQETSEDVAPPLPPFHFQRLLANLTSLRLRVSPGPSPAGPVFLTEVRLTSARPGLSPPASWVEICSCPTGYTGQFCESCAPGYKREMPQGGPYASCVPCTCNQHGTCDPNTGICVCSHHTEGPSCERCLPGFYGNPFAGQADDCQPCPCPGQSACTTIPESREVVCTHCPPGQRGRRCEVCDDGFFGDPLGLFGHPQPCHQCQCSGNVDPNAVGNCDPLSGHCLRCLHNTTGDHCEHCQEGFYGSALAPRPADKCMPCSCHPQGSVSEQMPCDPVTGQCSCLPHVTARDCSRCYPGFFDLQPGRGCRSCKCHPLGSQEDQCHPKTGQCTCRPGVTGQACDRCQLGFFGFSIKGCRACRCSPLGAASAQCHENGTCVCRPGFEGYKCDRCHDNFFLTADGTHCQQCPSCYALVKEEAAKLKARLTLTEGWLQGSDCGSPWGPLDILLGEAPRGDVYQGHHLLPGAREAFLEQMMSLEGAVKAAREQLQRLNKGARCAQAGSQKTCTQLADLEAVLESSEEEILHAAAILASLEIPQEGPSQPTKWSHLATEARALARSHRDTATKIAATAWRALLASNTSYALLWNLLEGRVALETQRDLEDRYQEVQAAQKALRTAVAEVLPEAESVLATVQQVGADTAPYLALLASPGALPQKSRAEDLGLKAKALEKTVASWQHMATEAARTLQTAAQATLRQTEPLTKLHQEARAALTQASSSVQAATVTVMGARTLLADLEGMKLQFPRPKDQAALQRKADSVSDRLLADTRKKTKQAERMLGNAAPLSSSAKKKGREAEVLAKDSAKLAKALLRERKQAHRRASRLTSQTQATLQQASQQVLASEARRQELEEAERVGAGLSEMEQQIRESRISLEKDIETLSELLARLGSLDTHQAPAQALNETQWALERLRLQLGSPGSLQRKLSLLEQESQQQELQIQGFESDLAEIRADKQNLEAILHSLPENCASWQ

(19) United States
(12) Patent Application Publication
(10) Pub. No.: US 2015/0337260 A1
(43) Pub. Date: Nov. 26, 2015

(54) Hybrid Matrix

(71) Applicant: THE SCHEPENS EYE RESEARCH INSTITUTE, INC., Boston, MA (US)

(72) Inventors: Michael Young, Essex, MA (US); Petr Y. Baranov, Somerville, MA (US)

(21) Appl. No.: 14/760,118

(22) PCT Filed: Jan. 10, 2014
(86) PCT No.: PCT/US14/11069

§ 371 Date: Jul. 9, 2015

Related U.S. Application Data
(60) Provisional application No. 61/750,993, filed on Jan. 10, 2013.

(51) Int. Cl. C12N 5/00; A61K 35/30

(52) U.S. Cl. CPC C12N 5/0068; A61K 35/30; C12N 2533/30; C12N 2533/20

(57) ABSTRACT

The present invention relates to poly(caprolactone)-oligonucleotide surfaces for cell ware, and methods of use thereof for culturing, and differentiating cells.

### FIELD OF THE DISCLOSURE

This application claims priority to and benefit of U.S. Provisional Application No. 61/750,993, filed on Jan. 10, 2013; the contents of which are hereby incorporated by reference in its entirety.

### FIELD OF THE DISCLOSURE

The present invention relates to poly(caprolactone)-oligonucleotide surfaces for cell ware and to poly(caprolactone)-oligonucleotide films for cell transplantation.

### BACKGROUND OF THE DISCLOSURE

Adherent cell cultures rely upon the proper attachment and spreading of cells on a surface. To alter the properties of cultured cells (e.g., adhesion, proliferation, morphology, and differentiation), the surface of tissue culture plastic (e.g., polystyrene or polypropylene) is altered in different ways. The most commonly used approaches include coating with extracellular matrix proteins, increasing hydrophilicity via oxygen plasma treatment, charging the surface, and incorporating bioactive peptides. However, these approaches do not change the stiffness properties of the substrate, which are important for growth and differentiation of specific cell types, such as photoreceptor precursor cells, neurons, or mesenchymal stem cells. Thus, there is a pressing need to develop improved cell culture scaffolds.

### SUMMARY OF THE DISCLOSURE

The present invention relates to hybrid poly(caprolactone)(PCL)—oligopeptide surfaces for cell culture, directed cell differentiation, and transplantation. The hybrid poly(caprolactone)(PCL)—oligopeptide scaffolds described herein provide advantages over existing PCL scaffolds. First, the hybrid poly(caprolactone)(PCL)—oligopeptide scaffolds described herein allow for storage or immediate use after manufacture without the need for additional modification prior to use. For example, cells are seeded on the hybrid surfaces immediately after manufacture without the need to provide any additional surface coating. The hybrid surfaces are also extremely stable, thereby enabling storage of the scaffolds prior to use. Second, the hybrid poly(caprolactone)(PCL)—oligopeptide surfaces lack allo- and xeno-material in the scaffold, e.g., fibronectin of human or bovine origin, thereby avoiding potential risk, e.g., rejection, during transplantation. Finally, the adhesive properties of the hybrid surfaces described herein may be modified by altering the concentration of incorporated oligopeptide, thereby affecting transplantation outcome.

Specifically, described herein is a hybrid matrix comprising PCL comprising embedded or interspersed oligopeptides containing an RGD motif (R: arginine; G: glycine; D: aspartic acid), referred to herein as RGD-oligopeptides. For example, the matrix comprises a mixture of PCL and RGD-oligopeptides. In some cases, the RGD-oligopeptide is covalently bound to PCL with a crosslinking agent such as glutaraldehyde.

Oligopeptides suitable for use in the hybrid matrix described herein include RGD-containing oligopeptides that mimic extracellular matrix (ECM) proteins. Examples of extracellular matrix proteins include vitronectin, laminin, fibronectin, and collagen. ECM-mimicking peptides, as used herein, refers to peptides that promote, induce, or maintain cell attachment, proliferation, spreading, viability, and/or differentiation. The ECM-mimicking peptides described herein contain the RGD motif. ECM-mimicking peptides can be identified by presence of one or more RGD motifs, and/or increased attachment, proliferation, spreading, viability, and/or differentiation of cells when cultured in presence of the ECM-mimicking peptides. Preferably, the RGD-containing oligopeptides used herein increase the adhesive properties, i.e., cell attachment, to PCL-coated surfaces.

The RGD-containing oligopeptides described herein are 5 to 10 amino acids, 5 to 15 amino acids, 5 to 20 amino acids, 5 to 25 amino acids, 5 to 30 amino acids, 5 to 35 amino acids, 5 to 40 amino acids, 5 to 50 amino acids, 5 to 60 amino acids, 5 to 70 amino acids, 5 to 80 amino acids, 5 to 90 amino acids, or 5 to 100 amino acids in length. Preferably, the RGD-oligopeptides are 5 to 20 amino acids in length, and even more preferably, 10 to 20 amino acids in length, for example, 15 amino acids in length.

The oligopeptides described herein are chemically synthesized using methods well-known in the art. Commercial vendors that produce custom peptides are known in the art, for example, GenScript (Piscataway, N.J.), Life Technologies (Grand Island, N.Y.), and Pierce/Thermo Fisher Scientific (Rockford, Ill.). Alternatively, the oligopeptides are translated in vitro from a nucleic acid expression vector. Recombinant DNA techniques for designing and constructing nucleic acid expression vectors are known in the art. For example, the nucleic acid expression vector comprises a nucleic acid sequence encoding an RGD-oligopeptide disclosed herein, preferably operably linked to a suitable promoter sequence that drives the expression of the peptide. Examples of in vitro transcription/translation systems include, but are not limited to, cell-free systems (i.e., rabbit reticulocyte lysate, insect cell lysate, wheat germ extracts, and E. coli extract); mammalian cells (i.e., transfection into human kidney 293 cells or HeLa cells); and commercially available kits (i.e., PURExpress® In Vitro Protein Synthesis Kit (New England Biolabs); TNT® Quick Coupled Transcription/Translation System (Promega); and EasyXpress Protein Synthesis Kit (Qiagen).

In some cases, the RGD-oligopeptide comprises an RGD-containing vitronectin-mimicking oligopeptide. For example, the RGD-containing vitronectin-mimicking oligopeptide is Synthemax®-II SC substrate (Corning, Cat. No. 3535XX1). The amino acid sequence of Synthemax®-II is KGGPQVTRGDVFTMP (SEQ ID NO: 1). Alternatively, the RGD-oligopeptide comprises an RGD-containing laminin-mimicking oligopeptide, e.g., an oligopeptide comprising the following amino acid sequence: KYGAASIKVAVSADR (SEQ ID NO: 2). In other cases, the RGD-oligopeptide comprises an RGD-containing fibronectin-mimicking oligopeptide, e.g., an oligopeptide comprising the following amino acid sequence: GRGDSPK (SEQ ID NO: 3) or KGGAVTGRGDSPASS (SEQ ID NO: 4).

For example, Synthemax®II-SC is present between 1% and 25% by weight of the hybrid matrix, e.g., between 2% and 24%, between 3% and 23%, between 4% and 22%, between 5% and 21%, between 6% and 20%, between 7% and 19%, between 8% and 18%, between 9% and 17%, between 10% and 16%, between 11% and 15%, between 12% and 14%, between 1% and 5%, between 5% and 10%, between 10% and 15%, between 15% and 20%, or between 20% and 25% of the final PCL preparation. Preferably, Synthemax® II-SC is present at the concentration, for example, 1 μg/ml, 2 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml or 100 μg/ml. Preferable, Synthemax®II-Sc is present at 3% or at 30 μg/ml.

The hybrid matrix is optionally in the form of a coating. For example, the hybrid matrix is suitable for application to a tissue culture plate, e.g., lab-ware, cell culture-ware, etc. In this manner, proliferation of cells on a massive scale is simplified, as there is no need to fix an additional film/coating on the bottom of the plate prior to seeding the cells.

Alternatively, the hybrid matrix is in the form of a wafer or a film, e.g., a thin film. For example, the hybrid matrix is in the form of a film suitable for application to a wafer, e.g., a silicon wafer. In some cases, the film is rough. For example, a thin film is manufactured with precise microtopography to produce grooves, pits, and/or pores. Alternatively, the film is smooth. The thickness of the film is controlled by manipulating the solution's PCL concentration and the spinning process. Preferably, the hybrid matrix does not comprise a fibronectin coating.

The hybrid matrix comprises a stiffness that is softer than tissue culture plastic, e.g., about 10 times softer, about 100 times softer, or about 1,000 times softer than tissue culture plastic. For example, the stiffness of culture plastic or culture glass is greater than 1 gigapascal (GPa), while the stiffness of the hybrid scaffold is manufactured to be from about 1 to about 150 megapascals (MPa), e.g., about 5, about 10, about 25, about 50, about 75, about 100, about 125, or about 150 MPa.

Preferably, the cells adhere to the coated tissue culture plate. For example, at least 50% of cells attach to the tissue culture plate, e.g., at least 55%, at least 60%, at least 65%, at least 70%, at least 75%, at least 80%, at least 85%, at least 90% or at least 95% of cells attach to the coated tissue culture plate.

Cells attach to the tissue culture plate for a duration of 24 hours to 2 weeks to allow for proliferation or differentiation of the cells, e.g., about 2 days, about 3 days, about 4 days, about 5 days, about 6 days, about 7 days, about 8 days, about 9 days, about 10 days, about 11 days about 12 days, about 13 days, or about 14 days. Preferably, the cells attach to the tissue culture plates for at least 7 days to allow for proliferation or differentiation.

The hybrid matrix is suitable for transplantation. For example, cells adhered to the hybrid matrix are transplanted into a subject to encourage the growth and/or differentiation of transplanted cells. In some cases, the tissue is damaged tissue, and transplanting the cells with or without the hybrid matrix achieves structural and functional recovery. For example, the cells with or without the hybrid matrix are delivered to degenerating or damaged retinal tissue.

The subject is preferably a mammal in need of such treatment, e.g., a subject that has tissue damage or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

In some aspects, the subject is suffering from a retinal degenerative disease or disorder. Examples of retinal degenerative disease or disorders include, but are not limited to, age-related macular degeneration and retinitis pigmentosa. In other aspects the subject is suffering from ocular tissue damage that causes loss or death of ocular cells, for example, physical trauma and ocular surgery.

A hybrid matrix is produced by dissolving PCL in an organic solvent and mixing a PCL solution with an RGD-oligopeptide. In some cases, the organic solvent is dichloromethane or chloroform. Subsequently, spin-casting for film manufacture or for coating of a surface is performed. The hybrid matrix is dried to evaporate the solvent. Optionally, the hybrid matrix is dried at 60° C. to create a smooth surface. The resulting hybrid matrix is then utilized for cell culture/transplantation.

A method of culturing one or more cells is carried out by allowing cells to grow on a hybrid matrix comprising PCL comprising embedded RGD-oligopeptide. This scaffold is utilized for cell types of ocular and non-ocular origin. Suitable ocular cells include, but are not limited to, retinal progenitor cells, retinal pigment epithelium cells, photoreceptor precursor cells, neurons (i.e., retinal neurons), limbal stem cells, and transient amplifying corneal epithelial cells. Suitable non-ocular cells include, but are not limited to keratinocytes, mesenchymal stem cells, and induced pluripotent stem cells (iPS). Preferably, the cells are human cells.

In some cases, proliferation of the cells is maintained on the hybrid matrix. Alternatively, proliferation of the cells is inhibited on the hybrid matrix. In other cases, cells differentiate on the hybrid matrix. For example, the hybrid matrix drives differentiation of human retinal progenitor cells toward photoreceptors, i.e., to mature retinal photoreceptor cells. Differentiation, as used herein, refers to the process of transforming one cell type to a different cell type. For example, the progenitor cells are differentiated into mature cells, such as retinal photoreceptor cells or retinal ganglion cells.

The differentiated cells cultured on the hybrid matrix preferably express at least one marker of a mature retinal photoreceptor cell. Examples of markers of retinal photoreceptor cells include, but are not limited to, Nr1, recoverin, and rhodopsin. Other markers of retinal photoreceptor cells are known in the art. Preferably, the differentiated cells exhibit increased expression of markers of mature cells, such as photoreceptor cell markers. By increased expression is meant expression that is 1%, 2%, 5%, 10%, 20%, 50% or 100%, 1-fold, 1.2 fold, 1.5-fold, 2-fold, 5-fold, or 10-fold increased compared to before differentiation or to undifferentiated cells. In other aspects, the differentiated cells exhibit decreased expression of markers of undifferentiated cells, such as stem cell or progenitor cell markers. Decreased expression, as used herein, refers to 1%, 2%, 5%, 10%, 20%, 25%, 50% or 100%, 1-fold, 1.2 fold, 1.5-fold, 2-fold, 5-fold, or 10-fold decreased expression with respect to before differentiation or to undifferentiated cells. Methods for detection of marker expression are known in the art, for example immunofluorescence and immunohistochemical analysis, or nucleic acid-based gene expression analysis, i.e. real time or quantitative PCR.

Optionally, the methods of the invention further comprise transplanting the cells into a damaged tissue or organ. For example, the scaffolds described herein are utilized for cell delivery into a damaged retina or cornea. The hybrid matrix described herein is suitable for cell culture/transplantation, as the matrix mediates cellular adhesion.

The cell culture substrates described herein perform as well or better than standard substrates but have significant advantages over standard substrates. Such advantages include ease of use, increased cell adhesion, and shelf stability. For example, incorporation of the RGD-containing oligopeptides (e.g., Synthemax®) increases adhesion approximately 6-9 fold compare to PCL without the oligopeptides. The oligopeptides are coated onto the cell attachment surface or incorporated into the cell culture substrate (e.g., interspersed or embedded throughout the substrate including on the cell attachment surface. From stability point, PCL with embedded oligopeptide has an advantage over coated PCL, since one can dry and store the scaffold (with embedded oligopeptides) at room temperature. Otherwise, oligopeptide-embedded or oligopeptide coated substrates/scaffolds perform similarly, e.g., with regard to differentiaiton, adhesion, and/or proliferation.

As described above, some agents of the invention are in the form of a polypeptide. Polypeptides or other agents are purified and/or isolated. Specifically, as used herein, an “isolated” or “purified polypeptide or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. Purified also defines a degree of sterility that is safe for administration to a human subject, e.g., lacking infectious or toxic agents.

Similarly, by “substantially pure” is meant a polypeptide that has been separated from the components that naturally accompany it. Typically, the polypeptides are substantially pure when they are at least 60%, 70%, 80%, 90%, 95%, or even 99%, by weight, free from the proteins and naturally-occurring organic molecules with they are naturally associated.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic diagram showing an exemplary method of making a hybrid matrix.

FIG. 2 is a photograph of two petri dishes coated with a Synthemax® II-SC/PCLhybrid matrix (left) or a thin polymer film (right).

FIG. 3 is a schematic of the design of the adhesion experiment described in Example 2.

FIG. 4 is a bar chart showing the percentage of cells adhered to PCL with different Synthemax® II concentrations after 30 min of incubation. A Student's T-test revealed no difference in adhesion for 30, 50 and 100 μg/ml preparations (p=0.57). However, adhesion in these groups was higher compared to 10 μg/m1 preparations (p<0.0001).

FIG. 5 is a bar chart showing the percentage of cells adhered to different substrates after 30 min of incubation. The substrates are as follows: plastic only (plastic), plastic coated with fibronectin (plastic+Fn); PCL only (PCL); PCL/fibronectin hybrid (PCL+Fn); and PCL/SynthemaxII-SC (PCL+S). Incorporation of Synthemax® II-SC significantly increased the adhesive properties of the PCL (p<0.0001), allowing for the same level of adhesion as on fibronectin-coated PCL (p=0.41).

FIG. 6 is a bar chart showing the increase in cell population size after 96 hours in culture on different substrates. The substrates are as follows: plastic only (plastic), plastic coated with fibronectin (plastic+Fn); PCL only (PCL); PCL/fibronectin hybrid (PCL+Fn); and PCL/SynthemaxII-SC (PCL+S). The hybrid scaffold inhibited the proliferation of human retinal progenitor cells (hRPCs) in the same manner as fibronectin-coated PCL (p=0.38).

FIG. 7 is a series of three histogram plots showing FACS analysis of three rod-specific markers: (A) Nr1; (B) Recoverin; and (C) Rhodopsin in hPRC after differentiation. PCL/fibronectin (blue); PCL/Synthemax II-SC (orange); before differentiation (red); and isotype control (black solid line).

FIG. 8 is a bar chart showing the quantification of a-wave or b-wave electroretinography recordings of retinas from rd1 mutant mice after transplantation of hPRCs differentiated on different substrates. The substrates include plastic only (Control); fibronectin-coated plastic (Fn); PCL/fibronectin (PCL-FN); PCL/SynthemaxII-SC (PCL-S).

FIG. 9 is two immunofluorescence images showing the integration of differentiated hPRCs into the outer nuclear layer of the retina of rd1 mutant mice 3 weeks after transplantation. (A) PCL/fibronectin; (B) PCL/SynthemaxII-SC.

### DETAILED DESCRIPTION OF THE INVENTION

Many advances have been made in an attempt to treat retinal degenerative diseases, such as age-related macular degeneration and retinitis pigmentosa. Irreversible loss of photoreceptors is common to both diseases, and currently, no restorative clinical treatment exists. It has been shown that progenitor cells and photoreceptor precursors isolated from the developing retina can rescue retinal structure and function upon transplantation. Effective tissue engineering strategies have been studied, and have shown that PCL induces mature photoreceptor differentiation from human retinal progenitor cells (hRPCs). However, poor adhesive properties limit its use; therefore additional surface modifications are required.

Adherent cell cultures rely upon the proper attachment and spreading of cells on a surface. To alter the properties of cultured cells (e.g., adhesion, proliferation, morphology, and differentiation), the surface of tissue culture plastic (e.g., polystyrene or polypropylene) may be altered in different ways. The most commonly used approaches are coating with extracellular matrix proteins, increasing hydrophilicity via oxygen plasma treatment, charging the surface, and incorporating bioactive peptides. However, these approaches do not change the stiffness properties of the substrate, which are important for growth and differentiation of specific cell types, such as photoreceptor precursor, neurons, or mesenchymal stem cells.

This disclosure describes the discovery that incorporation of RGD-containing oligopeptides that mimic certain ECM proteins into PCL coating to create a hybrid scaffold increases the attachment of cells in culture to PCL. Furthermore, this incorporation of RGD-oligopeptides with PCL retains the differentiation capabilities of PCL alone, thereby providing a novel hybrid matrix with improved adhesive properties. Cells cultured and/or differentiated on the hybrid matrix described herein are suitable for use in retinal transplantation to restore structure and function of damaged or degenerating retinal tissue.

The hybrid matrix comprises a stiffness that is softer than tissue culture plastic, e.g., about 10 times softer, about 100 times softer, or about 1,000 times softer than tissue culture plastic. For example, the stiffness of culture plastic or culture glass is greater than 1 gigapascal (GPa), while the stiffness of the hybrid scaffold is manufactured to be from about 1 to about 150 megapascals (MPa), e.g., about 5, about 10, about 25, about 50, about 75, about 100, about 125, or about 150 MPa.

A PCL film approach for transplantation of mice retinal progenitor cells was described previously (Redenti et al., 2008 J Ocul Biol Dis Inform, 1: 19-29); however, due to the hydrophobicity of PCL, this approach still requires additional coating (e.g., with poly-D-lysine, poly-L-lysine, fibronectin, laminin, collagen I, collagen IV, vitronectin, matrigel, etc.). PCL has been also widely utilized for culture, differentiation, and transplantation of neural precursors and mesenchymal stem cells of different species. However, prior to the invention described herein, the addition of gel compounds, such as gelatin, Matrigel or agarose was required to achieve a biomimetic microenvironment.

### Poly(Caprolactone) (PCL)

PCL is a biodegradable polyester with a low melting point of around 60° C. and a glass transition temperature of about −60° C. PCL imparts good water, oil, solvent and chlorine resistance to the polyurethane produced. PCL is prepared by ring opening polymerization of e-caprolactone using a catalyst such as stannous octoate. PCL is degraded by hydrolysis of its ester linkages in physiological conditions (such as in the human body). Thus, PCL is useful as an implantable biomaterial. In particular, it is useful for the preparation of long term implantable devices, due to its slow degradation which is even slower than that of polylactide. The molecular weight of PCL in the hybrid matrix described herein is about 70,000 to about 90,000; however, a lower molecular weight PCL is used for the manufacture of films.

The structural formula of PCL is as follows:

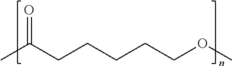

### RGD-Oligopeptide (R: Arginine; G: Glycine; D: Aspartic Acid)

Due to its widespread distribution in many organisms, its ability to address more than one cell adhesion receptor, and its role in cell anchoring, behavior, and survival, the RGD cell recognition motif is a peptide sequence that is utilized to promote cell adhesion on synthetic surfaces (Hersel, et al., 2003 Biomaterials, 24: 4385-4415). The RGD motif was originally identified as a minimal essential adhesion peptide in fibronectin; however, RGD motifs have also been identified in vitronectin, fibrinogen, von Willebrand factor, collagen, laminin, osteopontin, tenascin, bone sialoprotein, in membrane proteins, in viral and bacterial proteins, and in snake venoms (Pierschbacher M D and Ruoslahti E, 1984 Nature, 309: 30-33; Pfaff M, 1997 In: Eble J A, editor. Integrin-Ligand Interaction. Heidelberg: Springer-Verlag; p 101-121).

RGD-motifs are frequently present in the sequences of extra-cellular matrix proteins (ECM). These motifs are believed to play an important role in cell adhesion to the ECM. The RGD-oligopeptides of the present disclosure mimick ECM proteins and promote cell adhesion.

For example, the RGD-oligopeptide is a vitronectin-mimicking peptide. In some aspects, the RGD-oligopeptide is identical or similar to a sequence present in the human vitronectin protein. The RGD-oligopeptide may be 100%, 99%, 98%, 97%, 96%, 95%, 94%, 93%, 92%, 91%, 90%, 85%, 80%, 75%, 70%, 65%, 60%, 55%, or 50% identical to human vitronectin over the number of amino acids of the peptide. The amino acid sequence for human vitronectin can be found in GenBank Accession No. NP_{—}000629.3: (SEQ ID NO: 5)

MAPLRPLLILALLAWVALADQESCKGRCTEGFNVDKKCQCDELCSYYQSC
CTDYTAECKPQVTRGDVFTMPEDEYTVYDDGEEKNNATVHEQVGGPSLTS
DLQAQSKGNPEQTPVLKPEEEAPAPEVGASKPEGIDSRPETLHPGRPQPP
AEEELCSGKPFDAFTDLKNGSLFAFRGQYCYELDEKAVRPGYPKLIRDVW
GIEGPIDAAFTRINCQGKTYLFKGSQYWRFEDGVLDPDYPRNISDGFDGI
PDNVDAALALPAHSYSGRERVYFFKGKQYWEYQFQHQPSQEECEGSSLSA
VFEHFAMMQRDSWEDIFELLFWGRTSAGTRQPQFISRDWHGVPGQVDAAM
AGRIYISGMAPRPSLAKKQRFRHRNRKGYRSQRGHSRGRNQNSRRPSRAT
WLSLFSSEESNLGANNYDDYRMDWLVPATCEPIQSVFFFSGDKYYRVNLR
TRRVDTVDPPYPRSIAQYWLGCPAPGHL

In another aspect, the RGD-oligopeptide is a fibronectin-mimicking peptide. For example, the RGD-oligopeptide is identical or similar to a sequence present in the human fibronectin protein. The RGD-oligopeptide may be 100%, 99%, 98%, 97%, 96%, 95%, 94%, 93%, 92%, 91%, 90%, 85%, 80%, 75%, 70%, 65%, 60%, 55%, or 50% identical to human fibronectin over the number of amino acids of the peptide. The amino acid sequence for human fibronectin can be found in GenBank Accession No. NP_{—}002017.1: (SEQ ID NO: 6)

MLRGPGPGLLLLAVQCLGTAVPSTGASKSKRQAQQMVQPQSPVAVSQSKP
GCYDNGKHYQINQQWERTYLGNALVCTCYGGSRGFNCESKPEAEETCFDK
YTGNTYRVGDTYERPKDSMIWDCTCIGAGRGRISCTIANRCHEGGQSYKI
GDTWRRPHETGGYMLECVCLGNGKGEWTCKPIAEKCFDHAAGTSYVVGET
WEKPYQGWMMVDCTCLGEGSGRITCTSRNRCNDQDTRTSYRIGDTWSKKD
NRGNLLQCICTGNGRGEWKCERHTSVQTTSSGSGPFTDVRAAVYQPQPHP
QPPPYGHCVTDSGVVYSVGMQWLKTQGNKQMLCTCLGNGVSCQETAVTQT
YGGNSNGEPCVLPFTYNGRTFYSCTTEGRQDGHLWCSTTSNYEQDQKYSF
CTDHTVLVQTRGGNSNGALCHFPFLYNNHNYTDCTSEGRRDNMKWCGTTQ
NYDADQKFGFCPMAAHEEICTTNEGVMYRIGDQWDKQHDMGHMMRCTCVG
NGRGEWTCIAYSQLRDQCIVDDITYNVNDTFHKRHEEGHMLNCTCFGQGR
GRWKCDPVDQCQDSETGTFYQIGDSWEKYVHGVRYQCYCYGRGIGEWHCQ
PLQTYPSSSGPVEVFITETPSQPNSHPIQWNAPQPSHISKYILRWRPKNS
VGRWKEATIPGHLNSYTIKGLKPGVVYEGQLISIQQYGHQEVTRFDFTTT
STSTPVTSNTVTGETTPFSPLVATSESVTEITASSFVVSWVSASDTVSGF
RVEYELSEEGDEPQYLDLPSTATSVNIPDLLPGRKYIVNVYQISEDGEQS
LILSTSQTTAPDAPPDPTVDQVDDTSIVVRWSRPQAPITGYRIVYSPSVE
GSSTELNLPETANSVTLSDLQPGVQYNITIYAVEENQESTPVVIQQETTG
TPRSDTVPSPRDLQFVEVTDVKVTIMWTPPESAVTGYRVDVIPVNLPGEH
GQRLPISRNTFAEVTGLSPGVTYYFKVFAVSHGRESKPLTAQQTTKLDAP
TNLQFVNETDSTVLVRWTPPRAQITGYRLTVGLTRRGQPRQYNVGPSVSK
YPLRNLQPASEYTVSLVAIKGNQESPKATGVFTTLQPGSSIPPYNTEVTE
TTIVITWTPAPRIGFKLGVRPSQGGEAPREVTSDSGSIVVSGLTPGVEYV
YTIQVLRDGQERDAPIVNKVVTPLSPPTNLHLEANPDTGVLTVSWERSTT
PDITGYRITTTPTNGQQGNSLEEVVHADQSSCTFDNLSPGLEYNVSVYTV
KDDKESVPISDTIIPAVPPPTDLRFTNIGPDTMRVTWAPPPSIDLTNFLV
RYSPVKNEEDVAELSISPSDNAVVLTNLLPGTEYVVSVSSVYEQHESTPL
RGRQKTGLDSPTGIDFSDITANSFTVHWIAPRATITGYRIRHHPEHFSGR
PREDRVPHSRNSITLTNLTPGTEYVVSIVALNGREESPLLIGQQSTVSDV
PRDLEVVAATPTSLLISWDAPAVTVRYYRITYGETGGNSPVQEFTVPGSK
STATISGLKPGVDYTITVYAVTGRGDSPASSKPISINYRTEIDKPSQMQV
TDVQDNSISVKWLPSSSPVTGYRVTTTPKNGPGPTKTKTAGPDQTEMTIE
GLQPTVEYVVSVYAQNPSGESQPLVQTAVTNIDRPKGLAFTDVDVDSIKI
AWESPQGQVSRYRVTYSSPEDGIHELFPAPDGEEDTAELQGLRPGSEYTV
SVVALHDDMESQPLIGTQSTAIPAPTDLKFTQVTPTSLSAQWTPPNVQLT
GYRVRVTPKEKTGPMKEINLAPDSSSVVVSGLMVATKYEVSVYALKDTLT
SRPAQGVVTTLENVSPPRRARVTDATETTITISWRTKTETITGFQVDAVP
ANGQTPIQRTIKPDVRSYTITGLQPGTDYKIYLYTLNDNARSSPVVIDAS
TAIDAPSNLRFLATTPNSLLVSWQPPRARITGYIIKYEKPGSPPREVVPR
PRPGVTEATITGLEPGTEYTIYVIALKNNQKSEPLIGRKKTDELPQLVTL
PHPNLHGPEILDVPSTVQKTPFVTHPGYDTGNGIQLPGTSGQQPSVGQQM
IFEEHGFRRTTPPTTATPIRHRPRPYPPNVGQEALSQTTISWAPFQDTSE
YIISCHPVGTDEEPLQFRVPGTSTSATLTGLTRGATYNIIVEALKDQQRH
KVREEVVTVGNSVNEGLNQPTDDSCFDPYTVSHYAVGDEWERMSESGFKL
LCQCLGFGSGHFRCDSSRWCHDNGVNYKIGEKWDRQGENGQMMSCTCLGN
GKGEFKCDPHEATCYDDGKTYHVGEQWQKEYLGAICSCTCFGGQRGWRCD
NCRRPGGEPSPEGTTGQSYNQYSQRYHQRTNTNVNCPIECFMPLDVQADR
EDSRE

In another aspect, the RGD-oligopeptide in a laminin-mimicking peptide. Laminin is a trimeric protein that contains an cc-chain (LAMA1, LAMA2, LAMA3, LAMA4, and LAMAS), a β-chain (LAMB1, LAMB2, LAMB3, and LAMB4), and a γ-chain (LAMC1, LAMC2, and LAMC3). For example, the RGD-oligopeptide is identical or similar to a sequence present in any of the variants of human laminin. The RGD-oligopeptide may be 100%, 99%, 98%, 97%, 96%, 95%, 94%, 93%, 92%, 91%, 90%, 85%, 80%, 75%, 70%, 65%, 60%, 55%, or 50% identical to a human laminin variant over the number of amino acids of the peptide. The amino acid sequence for human laminin alpha 1 (LAMA1) (GenBank Accession No. NP_{—}005550.2) is as follows: (SEQ ID NO: 7)

MRGGVLLVLLLCVAAQCRQRGLFPAILNLASNAHISTNATCGEKGPEMFC
KLVEHVPGRPVRNPQCRICDGNSANPRERHPISHAIDGTNNWWQSPSIQN
GREYHWVTITLDLRQVFQVAYVIIKAANAPRPGNWILERSLDGTTFSPWQ
YYAVSDSECLSRYNITPRRGPPTYRADDEVICTSYYSRLVPLEHGEIHTS
LINGRPSADDLSPKLLEFTSARYIRLRLQRIRTLNADLMTLSHREPKELD
PIVTRRYYYSIKDISVGGMCICYGHASSCPWDETTKKLQCQCEHNTCGES
CNRCCPGYHQQPWRPGTVSSGNTCEACNCHNKAKDCYYDESVAKQKKSLN
TAGQFRGGGVCINCLQNTMGINCETCIDGYYRPHKVSPYEDEPCRPCNCD
PVGSLSSVCIKDDLHSDLHNGKQPGQCPCKEGYTGEKCDRCQLGYKDYPT
CVSCGCNPVGSASDEPCTGPCVCKENVEGKACDRCKPGFYNLKEKNPRGC
SECFCFGVSDVCSSLSWPVGQVNSMSGWLVTDLISPRKIPSQQDALGGRH
QVSINNTAVMQRLAPKYYWAAPEAYLGNKLTAFGGFLKYTVSYDIPVETV
DSNLMSHADVIIKGNGLTLSTQAEGLSLQPYEEYLNVVRLVPENFQDFHS
KRQIDRDQLMTVLANVTHLLIRANYNSAKMALYRLESVSLDIASSNAIDL
VVAADVEHCECPQGYTGTSCESCLSGYYRVDGILFGGICQPCECHGHAAE
CNVHGVCIACAHNTTGVHCEQCLPGFYGEPSRGTPGDCQPCACPLTIASN
NFSPTCHLNDGDEVVCDWCAPGYSGAWCERCADGYYGNPTVPGESCVPCD
CSGNVDPSEAGHCDSVTGECLKCLGNTDGAHCERCADGFYGDAVTAKNCR
ACECHVKGSHSAVCHLETGLCDCKPNVTGQQCDQCLHGYYGLDSGHGCRP
CNCSVAGSVSDGCTDEGQCHCVPGVAGKRCDRCAHGFYAYQDGSCTPCDC
PHTQNTCDPETGECVCPPHTQGVKCEECEDGHWGYDAEVGCQACNCSLVG
STHHRCDVVTGHCQCKSKFGGRACDQCSLGYRDFPDCVPCDCDLRGTSGD
ACNLEQGLCGCVEETGACPCKENVFGPQCNECREGTFALRADNPLGCSPC
FCSGLSHLCSELEDYVRTPVTLGSDQPLLRVVSQSNLRGTTEGVYYQAPD
FLLDAATVRQHIRAEPFYWRLPQQFQGDQLMAYGGKLKYSVAFYSLDGVG
TSNFEPQVLIKGGRIRKQVIYMDAPAPENGVRQEQEVAMRENFWKYFNSV
SEKPVTREDFMSVLSDIEYILIKASYGQGLQQSRISDISMEVGRKAEKLH
PEEEVASLLENCVCPPGTVGFSCQDCAPGYHRGKLPAGSDRGPRPLVAPC
VPCSCNNHSDTCDPNTGKCLNCGDNTAGDHCDVCTSGYYGKVTGSASDCA
LCACPHSPPASFSPTCVLEGDHDFRCDACLLGYEGKHCERCSSSYYGNPQ
TPGGSCQKCDCNPHGSVHGDCDRTSGQCVCRLGASGLRCDECEPRHILME
TDCVSCDDECVGVLLNDLDEIGDAVLSLNLTGIIPVPYGILSNLENTTKY
LQESLLKENMQKDLGKIKLEGVAEETDNLQKKLTRMLASTQKVNRATERI
FKESQDLAIAIERLQMSITEIMEKTTLNQTLDEDFLLPNSTLQNMQQNGT
SLLEIMQIRDFTQLHQNATLELKAAEDLLSQIQENYQKPLEELEVLKEAA
SHVLSKHNNELKAAEALVREAEAKMQESNHLLLMVNANLREFSDKKLHVQ
EEQNLTSELIVQGRGLIDAAAAQTDAVQDALEHLEDHQDKLLLWSAKIRH
HIDDLVMHMSQRNAVDLVYRAEDHAAEFQRLADVLYSGLENIRNVSLNAT
SAAYVHYNIQSLIEESEELARDAHRTVTETSLLSESLVSNGKAAVQRSSR
FLKEGNNLSRKLPGIALELSELRNKTNRFQENAVEITRQTNESLLILRAI
PKGIRDKGAKTKELATSASQSAVSTLRDVAGLSQELLNTSASLSRVNTTL
RETHQLLQDSTMATLLAGRKVKDVEIQANLLFDRLKPLKMLEENLSRNLS
EIKLLISQARKQAASIKVAVSADRDCIRAYQPQISSTNYNTLTLNVKTQE
PDNLLFYLGSSTASDFLAVEMRRGRVAFLWDLGSGSTRLEFPDFPIDDNR
WHSIHVARFGNIGSLSVKEMSSNQKSPTKTSKSPGTANVLDVNNSTLMFV
GGLGGQIKKSPAVKVTHFKGCLGEAFLNGKSIGLWNYIEREGKCRGCFGS
SQNEDPSFHFDGSGYSVVEKSLPATVTQIIMLFNTFSPNGLLLYLGSYGT
KDFLSIELFRGRVKVMTDLGSGPITLLTDRRYNNGTWYKIAFQRNRKQGV
LAVIDAYNTSNKETKQGETPGASSDLNRLDKDPIYVGGLPRSRVVRRGVT
TKSFVGCIKNLEISRSTFDLLRNSYGVRKGCLLEPIRSVSFLKGGYIELP
PKSLSPESEWLVTFATTNSSGIILAALGGDVEKRGDREEAHVPFFSVMLI
GGNIEVHVNPGDGTGLRKALLHAPTGTCSDGQAHSISLVRNRRIITVQLD
ENNPVEMKLGTLVESRTINVSNLYVGGIPEGEGTSLLTMRRSFHGCIKNL
IFNLELLDFNSAVGHEQVDLDTCWLSERPKLAPDAEDSKLLPEPRAFPEQ
CVVDAALEYVPGAHQFGLTQNSHFILPFNQSAVRKKLSVELSIRTFASSG
LIYYMAHQNQADYAVLQLHGGRLHFMFDLGKGRTKVSHPALLSDGKWHTV
KTDYVKRKGFITVDGRESPMVTVVGDGTMLDVEGLFYLGGLPSQYQARKI
GNITHSIPACIGDVTVNSKQLDKDSPVSAFTVNRCYAVAQEGTYFDGSGY
AALVKEGYKVQSDVNITLEFRTSSQNGVLLGISTAKVDAIGLELVDGKVL
FHVNNGAGRITAAYEPKTATVLCDGKWHTLQANKSKHRITLIVDGNAVGA
ESPHTQSTSVDTNNPIYVGGYPAGVKQKCLRSQTSFRGCLRKLALIKSPQ
VQSFDFSRAFELHGVFLHSCPGTES

The amino acid sequence for human laminin alpha 2 (LAMA2) (GenBank Accession No. NP_{—}0001417.2) is as follows: (SEQ ID NO: 8)

MPGAAGVLLLLLLSGGLGGVQAQRPQQQRQSQAHQQRGLFPAVLNLASNA
LITTNATCGEKGPEMYCKLVEHVPGQPVRNPQCRICNQNSSNPNQRHPIT
NAIDGKNTWWQSPSIKNGIEYHYVTITLDLQQVFQIAYVIVKAANSPRPG
NWILERSLDDVEYKPWQYHAVTDTECLTLYNIYPRTGPPSYAKDDEVICT
SFYSKIHPLENGEIHISLINGRPSADDPSPELLEFTSARYIRLRFQRIRT
LNADLMMFAHKDPREIDPIVTRRYYYSVKDISVGGMCICYGHARACPLDP
ATNKSRCECEHNTCGDSCDQCCPGFHQKPWRAGTFLTKTECEACNCHGKA
EECYYDENVARRNLSLNIRGKYIGGGVCINCTQNTAGINCETCTDGFFRP
KGVSPNYPRPCQPCHCDPIGSLNEVCVKDEKHARRGLAPGSCHCKTGFGG
VSCDRCARGYTGYPDCKACNCSGLGSKNEDPCFGPCICKENVEGGDCSRC
KSGFFNLQEDNWKGCDECFCSGVSNRCQSSYWTYGKIQDMSGWYLTDLPG
RIRVAPQQDDLDSPQQISISNAEARQALPHSYYWSAPAPYLGNKLPAVGG
QLTFTISYDLEEEEEDTERVLQLMIILEGNDLSISTAQDEVYLHPSEEHT
NVLLLKEESFTIHGTHFPVRRKEFMTVLANLKRVLLQITYSFGMDAIFRL
SSVNLESAVSYPTDGSIAAAVEVCQCPPGYTGSSCESCWPRHRRVNGTIF
GGICEPCQCFGHAESCDDVTGECLNCKDHTGGPYCDKCLPGFYGEPTKGT
SEDCQPCACPLNIPSNNFSPTCHLDRSLGLICDGCPVGYTGPRCERCAEG
YFGQPSVPGGSCQPCQCNDNLDFSIPGSCDSLSGSCLICKPGTTGRYCEL
CADGYFGDAVDAKNCQPCRCNAGGSFSEVCHSQTGQCECRANVQGQRCDK
CKAGTFGLQSARGCVPCNCNSFGSKSFDCEESGQCWCQPGVTGKKCDRCA
HGYFNFQEGGCTACECSHLGNNCDPKTGRCICPPNTIGEKCSKCAPNTWG
HSITTGCKACNCSTVGSLDFQCNVNTGQCNCHPKFSGAKCTECSRGHWNY
PRCNLCDCFLPGTDATTCDSETKKCSCSDQTGQCTCKVNVEGIHCDRCRP
GKFGLDAKNPLGCSSCYCFGTTTQCSEAKGLIRTWVTLKAEQTILPLVDE
ALQHTTTKGIVFQHPEIVAHMDLMREDLHLEPFYWKLPEQFEGKKLMAYG
GKLKYAIYFEAREETGFSTYNPQVIIRGGTPTHARIIVRHMAAPLIGQLT
RHEIEMTEKEWKYYGDDPRVHRTVTREDFLDILYDIHYILIKATYGNFMR
QSRISEISMEVAEQGRGTTMTPPADLIEKCDCPLGYSGLSCEACLPGFYR
LRSQPGGRTPGPTLGTCVPCQCNGHSSLCDPETSICQNCQHHTAGDFCER
CALGYYGIVKGLPNDCQQCACPLISSSNNFSPSCVAEGLDDYRCTACPRG
YEGQYCERCAPGYTGSPGNPGGSCQECECDPYGSLPVPCDPVTGFCTCRP
GATGRKCDGCKHWHAREGWECVFCGDECTGLLLGDLARLEQMVMSINLTG
PLPAPYKMLYGLENMTQELKHLLSPQRAPERLIQLAEGNLNTLVTEMNEL
LTRATKVTADGEQTGQDAERTNTRAKSLGEFIKELARDAEAVNEKAIKLN
ETLGTRDEAFERNLEGLQKEIDQMIKELRRKNLETQKEIAEDELVAAEAL
LKKVKKLFGESRGENEEMEKDLREKLADYKNKVDDAWDLLREATDKIREA
NRLFAVNQKNMTALEKKKEAVESGKRQIENTLKEGNDILDEANRLADEIN
SIIDYVEDIQTKLPPMSEELNDKIDDLSQEIKDRKLAEKVSQAESHAAQL
NDSSAVLDGILDEAKNISFNATAAFKAYSNIKDYIDEAEKVAKEAKDLAH
EATKLATGPRGLLKEDAKGCLQKSFRILNEAKKLANDVKENEDHLNGLKT
RIENADARNGDLLRTLNDTLGKLSAIPNDTAAKLQAVKDKARQANDTAKD
VLAQITELHQNLDGLKKNYNKLADSVAKTNAVVKDPSKNKIIADADATVK
NLEQEADRLIDKLKPIKELEDNLKKNISEIKELINQARKQANSIKVSVSS
GGDCIRTYKPEIKKGSYNNIVVNVKTAVADNLLFYLGSAKFIDFLAIEMR
KGKVSFLWDVGSGVGRVEYPDLTIDDSYWYRIVASRTGRNGTISVRALDG
PKASIVPSTHHSTSPPGYTILDVDANAMLFVGGLTGKLKKADAVRVITFT
GCMGETYFDNKPIGLWNFREKEGDCKGCTVSPQVEDSEGTIQFDGEGYAL
VSRPIRWYPNISTVMFKFRTFSSSALLMYLATRDLRDFMSVELTDGHIKV
SYDLGSGMASVVSNQNHNDGKWKSFTLSRIQKQANISIVDIDTNQEENIA
TSSSGNNFGLDLKADDKIYFGGLPTLRNLSMKARPEVNLKKYSGCLKDIE
ISRTPYNILSSPDYVGVTKGCSLENVYTVSFPKPGFVELSPVPIDVGTEI
NLSFSTKNESGIILLGSGGTPAPPRRKRRQTGQAYYVILLNRGRLEVHLS
TGARTMRKIVIRPEPNLFHDGREHSVHVERTRGIFTVQVDENRRYMQNLT
VEQPIEVKKLFVGGAPPEFQPSPLRNIPPFEGCIWNLVINSVPMDFARPV
SFKNADIGRCAHQKLREDEDGAAPAEIVIQPEPVPTPAFPTPTPVLTHGP
CAAESEPALLIGSKQFGLSRNSHIAIAFDDTKVKNRLTIELEVRTEAESG
LLFYMARINHADFATVQLRNGLPYFSYDLGSGDTHTMIPTKINDGQWHKI
KIMRSKQEGILYVDGASNRTISPKKADILDVVGMLYVGGLPINYTTRRIG
PVTYSIDGCVRNLHMAEAPADLEQPTSSFHVGTCFANAQRGTYFDGTGFA
KAVGGFKVGLDLLVEFEFRTTTTTGVLLGISSQKMDGMGIEMIDEKLMFH
VDNGAGRFTAVYDAGVPGHLCDGQWHKVTANKIKHRIELTVDGNQVEAQS
PNPASTSADTNDPVFVGGFPDDLKQFGLTTSIPFRGCIRSLKLTKGTGKP
LEVNFAKALELRGVQPVSCPAN

The amino acid sequence for human laminin alpha 3 (LAMA3) (GenBank Accession No. NP_{—}000218.2) is as follows: (SEQ ID NO: 9)

MPPAVRRSACSMGWLWIFGAALGQCLGYSSQQQRVPFLQPPGQSQLQASY
VEFRPSQGCSPGYYRDHKGLYTGRCVPCNCNGHSNQCQDGSGICVNCQHN
TAGEHCERCQEGYYGNAVHGSCRACPCPHTNSFATGCVVNGGDVRCSCKA
GYTGTQCERCAPGYFGNPQKFGGSCQPCSCNSNGQLGSCHPLTGDCINQE
PKDSSPAEECDDCDSCVMTLLNDLATMGEQLRLVKSQLQGLSASAGLLEQ
MRHMETQAKDLRNQLLNYRSAISNHGSKIEGLERELTDLNQEFETLQEKA
QVNSRKAQTLNNNVNRATQSAKELDVKIKNVIRNVHILLKQISGTDGEGN
NVPSGDFSREWAEAQRMMRELRNRNFGKHLREAEADKRESQLLLNRIRTW
QKTHQGENNGLANSIRDSLNEYEAKLSDLRARLQEAAAQAKQANGLNQEN
ERALGAIQRQVKEINSLQSDFTKYLTTADSSLLQTNIALQLMEKSQKEYE
KLAASLNEARQELSDKVRELSRSAGKTSLVEEAEKHARSLQELAKQLEEI
KRNASGDELVRCAVDAATAYENILNAIKAAEDAANRAASASESALQTVIK
EDLPRKAKTLSSNSDKLLNEAKMTQKKLKQEVSPALNNLQQTLNIVTVQK
EVIDTNLTTLRDGLHGIQRGDIDAMISSAKSMVRKANDITDEVLDGLNPI
QTDVERIKDTYGRTQNEDFKKALTDADNSVNKLTNKLPDLWRKIESINQQ
LLPLGNISDNMDRIRELIQQARDAASKVAVPMRFNGKSGVEVRLPNDLED
LKGYTSLSLFLQRPNSRENGGTENMFVMYLGNKDASRDYIGMAVVDGQLT
CVYNLGDREAELQVDQILTKSETKEAVMDRVKFQRIYQFARLNYTKGATS
SKPETPGVYDMDGRNSNTLLNLDPENVVFYVGGYPPDFKLPSRLSFPPYK
GCIELDDLNENVLSLYNFKKTFNLNTTEVEPCRRRKEESDKNYFEGTGYA
RVPTQPHAPIPTFGQTIQTTVDRGLLFFAENGDRFISLNIEDGKLMVRYK
LNSELPKERGVGDAINNGRDHSIQIKIGKLQKRMWINVDVQNTIIDGEVF
DFSTYYLGGIPIAIRERFNISTPAFRGCMKNLKKTSGVVRLNDTVGVTKK
CSEDWKLVRSASFSRGGQLSFTDLGLPPTDHLQASFGFQTFQPSGILLDH
QTWTRNLQVTLEDGYIELSTSDSGSPIFKSPQTYMDGLLHYVSVISDNSG
LRLLIDDQLLRNSKRLKHISSSRQSLRLGGSNFEGCISNVFVQRLSLSPE
VLDLTSNSLKRDVSLGGCSLNKPPFLMLLKGSTRFNKTKTFRINQLLQDT
PVASPRSVKVWQDACSPLPKTQANHGALQFGDIPTSHLLFKLPQELLKPR
SQFAVDMQTTSSRGLVFHTGTKNSFMALYLSKGRLVFALGTDGKKLRIKS
KEKCNDGKWHTVVFGHDGEKGRLVVDGLRAREGSLPGNSTISIRAPVYLG
SPPSGKPKSLPTNSFVGCLKNFQLDSKPLYTPSSSFGVSSCLGGPLEKGI
YFSEEGGHVVLAHSVLLGPEFKLVFSIRPRSLTGILIHIGSQPGKHLCVY
LEAGKVTASMDSGAGGTSTSVTPKQSLCDGQWHSVAVTIKQHILHLELDT
DSSYTAGQIPFPPASTQEPLHLGGAPANLTTLRIPVWKSFFGCLRNIHVN
HIPVPVTEALEVQGPVSLNGCPDQ

The amino acid sequence for human laminin alpha 4 (LAMA4) (GenBank Accession No. NP_{—}001098676.2) is as follows: (SEQ ID NO: 10)

MALSSAWRSVLPLWLLWSAACSRAASGDDNAFPFDIEGSSAVGRQDPPET
SEPRVALGRLPPAAEKCNAGFFHTLSGECVPCDCNGNSNECLDGSGYCVH
CQRNTTGEHCEKCLDGYIGDSIRGAPQFCQPCPCPLPHLANFAESCYRKN
GAVRCICNENYAGPNCERCAPGYYGNPLLIGSTCKKCDCSGNSDPNLIFE
DCDEVTGQCRNCLRNTTGFKCERCAPGYYGDARIAKNCAVCNCGGGPCDS
VTGECLEEGFEPPTGMDCPTISCDKCVWDLTDDLRLAALSIEEGKSGVLS
VSSGAAAHRHVNEINATIYLLKTKLSERENQYALRKIQINNAENTMKSLL
SDVEELVEKENQASRKGQLVQKESMDTINHASQLVEQAHDMRDKIQEINN
KMLYYGEEHELSPKEISEKLVLAQKMLEEIRSRQPFFTQRELVDEEADEA
YELLSQAESWQRLHNETRTLFPVVLEQLDDYNAKLSDLQEALDQALNYVR
DAEDMNRATAARQRDHEKQQERVREQMEVVNMSLSTSADSLTTPRLTLSE
LDDIIKNASGIYAEIDGAKSELQVKLSNLSNLSHDLVQEAIDHAQDLQQE
ANELSRKLHSSDMNGLVQKALDASNVYENIVNYVSEANETAEFALNTTDR
IYDAVSGIDTQIIYHKDESENLLNQARELQAKAESSSDEAVADTSRRVGG
ALARKSALKTRLSDAVKQLQAAERGDAQQRLGQSRLITEEANRTTMEVQQ
ATAPMANNLTNWSQNLQHFDSSAYNTAVNSARDAVRNLTEVVPQLLDQLR
TVEQKRPASNVSASIQRIRELIAQTRSVASKIQVSMMFDGQSAVEVHSRT
SMDDLKAFTSLSLYMKPPVKRPELTETADQFILYLGSKNAKKEYMGLAIK
NDNLVYVYNLGTKDVEIPLDSKPVSSWPAYFSIVKIERVGKHGKVFLTVP
SLSSTAEEKFIKKGEFSGDDSLLDLDPEDTVFYVGGVPSNFKLPTSLNLP
GFVGCLELATLNNDVISLYNFKHIYNMDPSTSVPCARDKLAFTQSRAASY
FFDGSGYAVVRDITRRGKFGQVTRFDIEVRTPADNGLILLMVNGSMFFRL
EMRNGYLHVFYDFGFSGGPVHLEDTLKKAQINDAKYHEISIIYHNDKKMI
LVVDRRHVKSMDNEKMKIPFTDIYIGGAPPEILQSRALRAHLPLDINFRG
CMKGFQFQKKDFNLLEQTETLGVGYGCPEDSLISRRAYFNGQSFIASIQK
ISFFDGFEGGFNFRTLQPNGLLFYYASGSDVFSISLDNGTVIMDVKGIKV
QSVDKQYNDGLSHFVISSVSPTRYELIVDKSRVGSKNPTKGKIEQTQASE
KKFYFGGSPISAQYANFTGCISNAYFTRVDRDVEVEDFQRYTEKVHTSLY
ECPIESSPLFLLHKKGKNLSKPKASQNKKGGKSKDAPSWDPVALKLPERN
TPRNSHCHLSNSPRAIEHAYQYGGTANSRQEFEHLKGDFGAKSQFSIRLR
TRSSHGMIFYVSDQEENDFMTLFLAHGRLVYMFNVGHKKLKIRSQEKYND
GLWHDVIFIRERSSGRLVIDGLRVLEESLPPTEATWKIKGPIYLGGVAPG
KAVKNVQINSIYSFSGCLSNLQLNGASITSASQTFSVTPCFEGPMETGTY
FSTEGGYVVLDESFNIGLKFEIAFEVRPRSSSGTLVHGHSVNGEYLNVHM
KNGQVIVKVNNGIRDFSTSVTPKQSLCDGRWHRITVIRDSNVVQLDVDSE
VNHVVGPLNPKPIDHREPVFVGGVPESLLTPRLAPSKPFTGCIRHFVIDG
HPVSFSKAALVSGAVSINSCPAA

The amino acid sequence for human laminin alpha 5 (LAMAS) (GenBank Accession No. NP_{—}005551.3) is as follows: (SEQ ID NO: 11)

MAKRLCAGSALCVRGPRGPAPLLLVGLALLGAARAREEAGGGFSLHPPYF
NLAEGARIAASATCGEEAPARGSPRPTEDLYCKLVGGPVAGGDPNQTIRG
QYCDICTAANSNKAHPASNAIDGTERWWQSPPLSRGLEYNEVNVTLDLGQ
VFHVAYVLIKFANSPRPDLWVLERSMDFGRTYQPWQFFASSKRDCLERFG
PQTLERITRDDAAICTTEYSRIVPLENGEIVVSLVNGRPGAMNFSYSPLL
REFTKATNVRLRFLRTNTLLGHLMGKALRDPTVTRRYYYSIKDISIGGRC
VCHGHADACDAKDPTDPFRLQCTCQHNTCGGTCDRCCPGFNQQPWKPATA
NSANECQSCNCYGHATDCYYDPEVDRRRASQSLDGTYQGGGVCIDCQHHT
TGVNCERCLPGFYRSPNHPLDSPHVCRRCNCESDFTDGTCEDLTGRCYCR
PNFSGERCDVCAEGFTGFPSCYPTPSSSNDTREQVLPAGQIVNCDCSAAG
TQGNACRKDPRVGRCLCKPNFQGTHCELCAPGFYGPGCQPCQCSSPGVAD
DRCDPDTGQCRCRVGFEGATCDRCAPGYFHFPLCQLCGCSPAGTLPEGCD
EAGRCLCQPEFAGPHCDRCRPGYHGFPNCQACTCDPRGALDQLCGAGGLC
RCRPGYTGTACQECSPGFHGFPSCVPCHCSAEGSLHAACDPRSGQCSCRP
RVTGLRCDTCVPGAYNFPYCEAGSCHPAGLAPVDPALPEAQVPCMCRAHV
EGPSCDRCKPGFWGLSPSNPEGCTRCSCDLRGTLGGVAECQPGTGQCFCK
PHVCGQACASCKDGFFGLDQADYFGCRSCRCDIGGALGQSCEPRTGVCRC
RPNTQGPTCSEPARDHYLPDLHHLRLELEEAATPEGHAVRFGFNPLEFEN
FSWRGYAQMAPVQPRIVARLNLTSPDLFWLVFRYVNRGAMSVSGRVSVRE
EGRSATCANCTAQSQPVAFPPSTEPAFITVPQRGFGEPFVLNPGTWALRV
EAEGVLLDYVVLLPSAYYEAALLQLRVTEACTYRPSAQQSGDNCLLYTHL
PLDGFPSAAGLEALCRQDNSLPRPCPTEQLSPSHPPLITCTGSDVDVQLQ
VAVPQPGRYALVVEYANEDARQEVGVAVHTPQRAPQQGLLSLHPCLYSTL
CRGTARDTQDHLAVFHLDSEASVRLTAEQARFFLHGVTLVPIEEFSPEFV
EPRVSCISSHGAFGPNSAACLPSRFPKPPQPIILRDCQVIPLPPGLPLTH
AQDLTPAMSPAGPRPRPPTAVDPDAEPTLLREPQATVVFTTHVPTLGRYA
FLLHGYQPAHPTFPVEVLINAGRVWQGHANASFCPHGYGCRTLVVCEGQA
LLDVTHSELTVTVRVPKGRWLWLDYVLVVPENVYSFGYLREEPLDKSYDF
ISHCAAQGYHISPSSSSLFCRNAAASLSLFYNNGARPCGCHEVGATGPTC
EPFGGQCPCHAHVIGRDCSRCATGYWGFPNCRPCDCGARLCDELTGQCIC
PPRTIPPDCLLCQPQTFGCHPLVGCEECNCSGPGIQELTDPTCDTDSGQC
KCRPNVTGRRCDTCSPGFHGYPRCRPCDCHEAGTAPGVCDPLTGQCYCKE
NVQGPKCDQCSLGTFSLDAANPKGCTRCFCFGATERCRSSSYTRQEFVDM
EGWVLLSTDRQVVPHERQPGTEMLRADLRHVPEAVPEAFPELYWQAPPSY
LGDRVSSYGGTLRYELHSETQRGDVFVPMESRPDVVLQGNQMSITFLEPA
YPTPGHVHRGQLQLVEGNFRHTETRNTVSREELMMVLASLEQLQIRALFS
QISSAVFLRRVALEVASPAGQGALASNVELCLCPASYRGDSCQECAPGFY
RDVKGLFLGRCVPCQCHGHSDRCLPGSGVCVDCQHNTEGAHCERCQAGFV
SSRDDPSAPCVSCPCPLSVPSNNFAEGCVLRGGRTQCLCKPGYAGASCER
CAPGFFGNPLVLGSSCQPCDCSGNGDPNLLFSDCDPLTGACRGCLRHTTG
PRCEICAPGFYGNALLPGNCTRCDCTPCGTEACDPHSGHCLCKAGVTGRR
CDRCQEGHFGFDGCGGCRPCACGPAAEGSECHPQSGQCHCRPGTMGPQCR
ECAPGYWGLPEQGCRRCQCPGGRCDPHTGRCNCPPGLSGERCDTCSQQHQ
VPVPGGPVGHSIHCEVCDHCVVLLLDDLERAGALLPAIHEQLRGINASSM
AWARLHRLNASIADLQSQLRSPLGPRHETAQQLEVLEQQSTSLGQDARRL
GGQAVGTRDQASQLLAGTEATLGHAKTLLAAIRAVDRTLSELMSQTGHLG
LANASAPSGEQLLRTLAEVERLLWEMRARDLGAPQAAAEAELAAAQRLLA
RVQEQLSSLWEENQALATQTRDRLAQHEAGLMDLREALNRAVDATREAQE
LNSRNQERLEEALQRKQELSRDNATLQATLHAARDTLASVFRLLHSLDQA
KEELERLAASLDGARTPLLQRMQTFSPAGSKLRLVEAAEAHAQQLGQLAL
NLSSIILDVNQDRLTQRAIEASNAYSRILQAVQAAEDAAGQALQQADHTW
ATVVRQGLVDRAQQLLANSTALEEAMLQEQQRLGLVWAALQGARTQLRDV
RAKKDQLEAHIQAAQAMLAMDTDETSKKIAHAKAVAAEAQDTATRVQSQL
QAMQENVERWQGQYEGLRGQDLGQAVLDAGHSVSTLEKTLPQLLAKLSIL
ENRGVHNASLALSASIGRVRELIAQARGAASKVKVPMKFNGRSGVQLRTP
RDLADLAAYTALKFYLQGPEPEPGQGTEDRFVMYMGSRQATGDYMGVSLR
DKKVHWVYQLGEAGPAVLSIDEDIGEQFAAVSLDRTLQFGHMSVTVERQM
IQETKGDTVAPGAEGLLNLRPDDFVFYVGGYPSTFTPPPLLRFPGYRGCI
EMDTLNEEVVSLYNFERTFQLDTAVDRPCARSKSTGDPWLTDGSYLDGTG
FARISFDSQISTTKRFEQELRLVSYSGVLFFLKQQSQFLCLAVQEGSLVL
LYDFGAGLKKAVPLQPPPPLTSASKAIQVFLLGGSRKRVLVRVERATVYS
VEQDNDLELADAYYLGGVPPDQLPPSLRRLFPTGGSVRGCVKGIKALGKY
VDLKRLNTTGVSAGCTADLLVGRAMTFHGHGFLRLALSNVAPLTGNVYSG
FGFHSAQDSALLYYRASPDGLCQVSLQQGRVSLQLLRTEVKTQAGFADGA
PHYVAFYSNATGVWLYVDDQLQQMKPHRGPPPELQPQPEGPPRLLLGGLP
ESGTIYNFSGCISNVFVQRLLGPQRVFDLQQNLGSVNVSTGCAPALQAQT
PGLGPRGLQATARKASRRSRQPARHPACMLPPHLRTTRDSYQFGGSLSSH
LEFVGILARHRNWPSLSMHVLPRSSRGLLLFTARLRPGSPSLALFLSNGH
FVAQMEGLGTRLRAQSRQRSRPGRWHKVSVRWEKNRILLVTDGARAWSQE
GPHRQHQGAEHPQPHTLFVGGLPASSHSSKLPVTVGFSGCVKRLRLHGRP
LGAPTRMAGVTPCILGPLEAGLFFPGSGGVITLDLPGATLPDVGLELEVR
PLAVTGLIFHLGQARTPPYLQLQVTEKQVLLRADDGAGEFSTSVTRPSVL
CDGQWHRLAVMKSGNVLRLEVDAQSNHTVGPLLAAAAGAPAPLYLGGLPE
PMAVQPWPPAYCGCMRRLAVNRSPVAMTRSVEVHGAVGASGCPAA

The amino acid sequence for human laminin beta 1 (LAMB1) (GenBank Accession No. NP_{—}002282.2) is as follows: (SEQ ID NO: 12)

MGLLQLLAFSFLALCRARVRAQEPEFSYGCAEGSCYPATGDLLIGRAQKL
SVTSTCGLHKPEPYCIVSHLQEDKKCFICNSQDPYHETLNPDSHLIENVV
TTFAPNRLKIWWQSENGVENVTIQLDLEAEFHFTHLIMTFKTFRPAAMLI
ERSSDFGKTWGVYRYFAYDCEASFPGISTGPMKKVDDIICDSRYSDIEPS
TEGEVIFRALDPAFKIEDPYSPRIQNLLKITNLRIKFVKLHTLGDNLLDS
RMEIREKYYYAVYDMVVRGNCFCYGHASECAPVDGFNEEVEGMVHGHCMC
RHNTKGLNCELCMDFYHDLPWRPAEGRNSNACKKCNCNEHSISCHFDMAV
YLATGNVSGGVCDDCQHNTMGRNCEQCKPFYYQHPERDIRDPNFCERCTC
DPAGSQNEGICDSYTDFSTGLIAGQCRCKLNVEGEHCDVCKEGFYDLSSE
DPFGCKSCACNPLGTIPGGNPCDSETGHCYCKRLVTGQHCDQCLPEHWGL
SNDLDGCRPCDCDLGGALNNSCFAESGQCSCRPHMIGRQCNEVEPGYYFA
TLDHYLYEAEEANLGPGVSIVERQYIQDRIPSWTGAGFVRVPEGAYLEFF
IDNIPYSMEYDILIRYEPQLPDHWEKAVITVQRPGRIPTSSRCGNTIPDD
DNQVVSLSPGSRYVVLPRPVCFEKGTNYTVRLELPQYTSSDSDVESPYTL
IDSLVLMPYCKSLDIFTVGGSGDGVVTNSAWETFQRYRCLENSRSVVKTP
MTDVCRNIIFSISALLHQTGLACECDPQGSLSSVCDPNGGQCQCRPNVVG
RTCNRCAPGTFGFGPSGCKPCECHLQGSVNAFCNPVTGQCHCFQGVYARQ
CDRCLPGHWGFPSCQPCQCNGHADDCDPVTGECLNCQDYTMGHNCERCLA
GYYGDPIIGSGDHCRPCPCPDGPDSGRQFARSCYQDPVTLQLACVCDPGY
IGSRCDDCASGYFGNPSEVGGSCQPCQCHNNIDTTDPEACDKETGRCLKC
LYHTEGEHCQFCRFGYYGDALQQDCRKCVCNYLGTVQEHCNGSDCQCDKA
TGQCLCLPNVIGQNCDRCAPNTWQLASGTGCDPCNCNAAHSFGPSCNEFT
GQCQCMPGFGGRTCSECQELFWGDPDVECRACDCDPRGIETPQCDQSTGQ
CVCVEGVEGPRCDKCTRGYSGVFPDCTPCHQCFALWDVIIAELTNRTHRF
LEKAKALKISGVIGPYRETVDSVERKVSEIKDILAQSPAAEPLKNIGNLF
EEAEKLIKDVTEMMAQVEVKLSDTTSQSNSTAKELDSLQTEAESLDNTVK
ELAEQLEFIKNSDIRGALDSITKYFQMSLEAEERVNASTTEPNSTVEQSA
LMRDRVEDVMMERESQFKEKQEEQARLLDELAGKLQSLDLSAAAEMTCGT
PPGASCSETECGGPNCRTDEGERKCGGPGCGGLVTVAHNAWQKAMDLDQD
VLSALAEVEQLSKMVSEAKLRADEAKQSAEDILLKTNATKEKMDKSNEEL
RNLIKQIRNFLTQDSADLDSIEAVANEVLKMEMPSTPQQLQNLTEDIRER
VESLSQVEVILQHSAADIARAEMLLEEAKRASKSATDVKVTADMVKEALE
EAEKAQVAAEKAIKQADEDIQGTQNLLTSIESETAASEETLFNASQRISE
LERNVEELKRKAAQNSGEAEYIEKVVYTVKQSAEDVKKTLDGELDEKYKK
VENLIAKKTEESADARRKAEMLQNEAKTLLAQANSKLQLLKDLERKYEDN
QRYLEDKAQELARLEGEVRSLLKDISQKVAVYSTCL

The amino acid sequence for human laminin beta 2 (LAMB2) (GenBank Accession No. NP_{—}002283.3) is as follows: (SEQ ID NO: 13)

MELTSRERGRGQPLPWELRLGLLLSVLAATLAQAPAPDVPGCSRGSCYPA
TGDLLVGRADRLTASSTCGLNGPQPYCIVSHLQDEKKCFLCDSRRPFSAR
DNPHSHRIQNVVTSFAPQRRAAWWQSENGIPAVTIQLDLEAEFHFTHLIM
TFKTFRPAAMLVERSADFGRTWHVYRYFSYDCGADFPGVPLAPPRHWDDV
VCESRYSEIEPSTEGEVIYRVLDPAIPIPDPYSSRIQNLLKITNLRVNLT
RLHTLGDNLLDPRREIREKYYYALYELVVRGNCFCYGHASECAPAPGAPA
HAEGMVHGACICKHNTRGLNCEQCQDFYRDLPWRPAEDGHSHACRKCECH
GHTHSCHFDMAVYLASGNVSGGVCDGCQHNTAGRHCELCRPFFYRDPTKD
LRDPAVCRSCDCDPMGSQDGGRCDSHDDPALGLVSGQCRCKEHVVGTRCQ
QCRDGFFGLSISDRLGCRRCQCNARGTVPGSTPCDPNSGSCYCKRLVTGR
GCDRCLPGHWGLSHDLLGCRPCDCDVGGALDPQCDEGTGQCHCRQHMVGR
RCEQVQPGYFRPFLDHLIWEAEDTRGQVLDVVERLVTPGETPSWTGSGFV
RLQEGQTLEFLVASVPKAMDYDLLLRLEPQVPEQWAELELIVQRPGPVPA
HSLCGHLVPKDDRIQGTLQPHARYLIFPNPVCLEPGISYKLHLKLVRTGG
SAQPETPYSGPGLLIDSLVLLPRVLVLEMFSGGDAAALERQATFERYQCH
EEGLVPSKTSPSEACAPLLISLSTLIYNGALPCQCNPQGSLSSECNPHGG
QCLCKPGVVGRRCDLCAPGYYGFGPTGCQACQCSHEGALSSLCEKTSGQC
LCRTGAFGLRCDRCQRGQWGFPSCRPCVCNGHADECNTHTGACLGCRDHT
GGEHCERCIAGFHGDPRLPYGGQCRPCPCPEGPGSQRHFATSCHQDEYSQ
QIVCHCRAGYTGLRCEACAPGHFGDPSRPGGRCQLCECSGNIDPMDPDAC
DPHTGQCLRCLHHTEGPHCAHCKPGFHGQAARQSCHRCTCNLLGTNPQQC
PSPDQCHCDPSSGQCPCLPNVQGPSCDRCAPNFWNLTSGHGCQPCACHPS
RARGPTCNEFTGQCHCRAGFGGRTCSECQELHWGDPGLQCHACDCDSRGI
DTPQCHRFTGHCSCRPGVSGVRCDQCARGFSGIFPACHPCHACFGDWDRV
VQDLAARTQRLEQRAQELQQTGVLGAFESSFWHMQEKLGIVQGIVGARNT
SAASTAQLVEATEELRREIGEATEHLTQLEADLTDVQDENFNANHALSGL
ERDRLALNLTLRQLDQHLDLLKHSNFLGAYDSIRHAHSQSAEAERRANTS
ALAVPSPVSNSASARHRTEALMDAQKEDFNSKHMANQRALGKLSAHTHTL
SLTDINELVCGAPGDAPCATSPCGGAGCRDEDGQPRCGGLSCNGAAATAD
LALGRARHTQAELQRALAEGGSILSRVAETRRQASEAQQRAQAALDKANA
SRGQVEQANQELQELIQSVKDFLNQEGADPDSIEMVATRVLELSIPASAE
QIQHLAGAIAERVRSLADVDAILARTVGDVRRAEQLLQDARRARSWAEDE
KQKAETVQAALEEAQRAQGIAQGAIRGAVADTRDTEQTLYQVQERMAGAE
RALSSAGERARQLDALLEALKLKRAGNSLAASTAEETAGSAQGRAQEAEQ
LLRGPLGDQYQTVKALAERKAQGVLAAQARAEQLRDEARDLLQAAQDKLQ
RLQELEGTYEENERALESKAAQLDGLEARMRSVLQAINLQVQIYNTCQ

The amino acid sequence for human laminin beta 3 (LAMB3) (GenBank Accession No. NP_{—}000218.2) is as follows: (SEQ ID NO: 14)

MRPFFLLCFALPGLLHAQQACSRGACYPPVGDLLVGRTRFLRASSTCGLT
KPETYCTQYGEWQMKCCKCDSRQPHNYYSHRVENVASSSGPMRWWQSQND
VNPVSLQLDLDRRFQLQEVMMEFQGPMPAGMLIERSSDFGKTWRVYQYLA
ADCTSTFPRVRQGRPQSWQDVRCQSLPQRPNARLNGGKVQLNLMDLVSGI
PATQSQKIQEVGEITNLRVNFTRLAPVPQRGYHPPSAYYAVSQLRLQGSC
FCHGHADRCAPKPGASAGPSTAVQVHDVCVCQHNTAGPNCERCAPFYNNR
PWRPAEGQDAHECQRCDCNGHSETCHFDPAVFAASQGAYGGVCDNCRDHT
EGKNCERCQLHYFRNRRPGASIQETCISCECDPDGAVPGAPCDPVTGQCV
CKEHVQGERCDLCKPGFTGLTYANPQGCHRCDCNILGSRRDMPCDEESGR
CLCLPNVVGPKCDQCAPYHWKLASGQGCEPCACDPHNSLSPQCNQFTGQC
PCREGFGGLMCSAAAIRQCPDRTYGDVATGCRACDCDFRGTEGPGCDKAS
GRCLCRPGLTGPRCDQCQRGYCNRYPVCVACHPCFQTYDADLREQALRFG
RLRNATASLWSGPGLEDRGLASRILDAKSKIEQIRAVLSSPAVTEQEVAQ
VASAILSLRRTLQGLQLDLPLEEETLSLPRDLESLDRSFNGLLTMYQRKR
EQFEKISSADPSGAFRMLSTAYEQSAQAAQQVSDSSRLLDQLRDSRREAE
RLVRQAGGGGGTGSPKLVALRLEMSSLPDLTPTFNKLCGNSRQMACTPIS
CPGELCPQDNGTACGSRCRGVLPRAGGAFLMAGQVAEQLRGFNAQLQRTR
QMIRAAEESASQIQSSAQRLETQVSASRSQMEEDVRRTRLLIQQVRDFLT
DPDTDAATIQEVSEAVLALWLPTDSATVLQKMNEIQAIAARLPNVDLVLS
QTKQDIARARRLQAEAEEARSRAHAVEGQVEDVVGNLRQGTVALQEAQDT
MQGTSRSLRLIQDRVAEVQQVLRPAEKLVTSMTKQLGDFWTRMEELRHQA
RQQGAEAVQAQQLAEGASEQALSAQEGFERIKQKYAELKDRLGQSSMLGE
QGARIQSVKTEAEELFGETMEMMDRMKDMELELLRGSQAIMLRSADLTGL
EKRVEQIRDHINGRVLYYATCK

The amino acid sequence for human laminin beta 4 (LAMB4) (GenBank Accession No. NP_{—}031382.2) is as follows: (SEQ ID NO: 15)

MQFQLTLFLHLGWLSYSKAQDDCNRGACHPTTGDLLVGRNTQLMASSTCG
LSRAQKYCILSYLEGEQKCFICDSRFPYDPYDQPNSHTIENVIVSFEPDR
EKKWWQSENGLDHVSIRLDLEALFRFSHLILTFKTFRPAAMLVERSTDYG
HNWKVFKYFAKDCATSFPNITSGQAQGVGDIVCDSKYSDIEPSTGGEVVL
KVLDPSFEIENPYSPYIQDLVTLTNLRINFTKLHTLGDALLGRRQNDSLD
KYYYALYEMIVRGSCFCNGHASECRPMQKMRGDVFSPPGMVHGQCVCQHN
TDGPNCERCKDFFQDAPWRPAADLQDNACRSCSCNSHSSRCHFDMTTYLA
SGGLSGGVCEDCQHNTEGQHCDRCRPLFYRDPLKTISDPYACIPCECDPD
GTISGGICVSHSDPALGSVAGQCLCKENVEGAKCDQCKPNHYGLSATDPL
GCQPCDCNPLGSLPFLTCDVDTGQCLCLSYVTGAHCEECTVGYWGLGNHL
HGCSPCDCDIGGAYSNVCSPKNGQCECRPHVTGRSCSEPAPGYFFAPLNF
YLYEAEEATTLQGLAPLGSETFGQSPAVHVVLGEPVPGNPVTWTGPGFAR
VLPGAGLRFAVNNIPFPVDFTIAIHYETQSAADWTVQIVVNPPGGSEHCI
PKTLQSKPQSFALPAATRIMLLPTPICLEPDVQYSIDVYFSQPLQGESHA
HSHVLVDSLGLIPQINSLENFCSKQDLDEYQLHNCVEIASAMGPQVLPGA
CERLIISMSAKLHDGAVACKCHPQGSVGSSCSRLGGQCQCKPLVVGRCCD
RCSTGSYDLGHHGCHPCHCHPQGSKDTVCDQVTGQCPCHGEVSGRRCDRC
LAGYFGFPSCHPCPCNRFAELCDPETGSCFNCGGFTTGRNCERCIDGYYG
NPSSGQPCRPCLCPDDPSSNQYFAHSCYQNLWSSDVICNCLQGYTGTQCG
ECSTGFYGNPRISGAPCQPCACNNNIDVTDPESCSRVTGECLRCLHNTQG
ANCQLCKPGHYGSALNQTCRRCSCHASGVSPMECPPGGGACLCDPVTGAC
PCLPNVTGLACDRCADGYWNLVPGRGCQSCDCDPRTSQSSHCDQLTGQCP
CKLGYGGKRCSECQENYYGDPPGRCIPCDCNRAGTQKPICDPDTGMCRCR
EGVSGQRCDRCARGHSQEFPTCLQCHLCFDQWDHTISSLSKAVQGLMRLA
ANMEDKRETLPVCEADFKDLRGNVSEIERILKHPVFPSGKFLKVKDYHDS
VRRQIMQLNEQLKAVYEFQDLKDTIERAKNEADLLLEDLQEEIDLQSSVL
NASIADSSENIKKYYHISSSAEKKINETSSTINTSANTRNDLLTILDTLT
SKGNLSLERLKQIKIPDIQILNEKVCGDPGNVPCVPLPCGGALCTGRKGH
RKCRGPGCHGSLTLSTNALQKAQEAKSIIRNLDKQVRGLKNQIESISEQA
EVSKNNALQLREKLGNIRNQSDSEEENINLFIKKVKNFLLEENVPPEDIE
KVANGVLDIHLPIPSQNLTDELVKIQKHMQLCEDYRTDENRLNEEADGAQ
KLLVKAKAAEKAANILLNLDKTLNQLQQAQITQGRANSTITQLTANITKI
KKNVLQAENQTREMKSELELAKQRSGLEDGLSLLQTKLQRHQDHAVNAKV
QAESAQHQAGSLEKEFVELKKQYAILQRKTSTTGLTKETLGKVKQLKDAA
EKLAGDTEAKIRRITDLERKIQDLNLSRQAKADQLRILEDQVVAIKNEIV
EQEKKYARCYS

The amino acid sequence for human laminin gamma 1 (LAMC1) (GenBank Accession No. NP_{—}002284.3) is as follows: (SEQ ID NO: 16)

MRGSHRAAPALRPRGRLWPVLAVLAAAAAAGCAQAAMDECTDEGGRPQRC
MPEFVNAAFNVTVVATNTCGTPPEEYCVQTGVTGVTKSCHLCDAGQPHLQ
HGAAFLTDYNNQADTTWWQSQTMLAGVQYPSSINLTLHLGKAFDITYVRL
KFHTSRPESFAIYKRTREDGPWIPYQYYSGSCENTYSKANRGFIRTGGDE
QQALCTDEFSDISPLTGGNVAFSTLEGRPSAYNFDNSPVLQEWVTATDIR
VTLNRLNTFGDEVFNDPKVLKSYYYAISDFAVGGRCKCNGHASECMKNEF
DKLVCNCKHNTYGVDCEKCLPFFNDRPWRRATAESASECLPCDCNGRSQE
CYFDPELYRSTGHGGHCTNCQDNTDGAHCERCRENFFRLGNNEACSSCHC
SPVGSLSTQCDSYGRCSCKPGVMGDKCDRCQPGFHSLTEAGCRPCSCDPS
GSIDECNIETGRCVCKDNVEGFNCERCKPGFFNLESSNPRGCTPCFCFGH
SSVCTNAVGYSVYSISSTFQIDEDGWRAEQRDGSEASLEWSSERQDIAVI
SDSYFPRYFIAPAKFLGKQVLSYGQNLSFSFRVDRRDTRLSAEDLVLEGA
GLRVSVPLIAQGNSYPSETTVKYVFRLHEATDYPWRPALTPFEFQKLLNN
LTSIKIRGTYSERSAGYLDDVTLASARPGPGVPATWVESCTCPVGYGGQF
CEMCLSGYRRETPNLGPYSPCVLCACNGHSETCDPETGVCNCRDNTAGPH
CEKCSDGYYGDSTAGTSSDCQPCPCPGGSSCAVVPKTKEVVCTNCPTGTT
GKRCELCDDGYFGDPLGRNGPVRLCRLCQCSDNIDPNAVGNCNRLTGECL
KCIYNTAGFYCDRCKDGFFGNPLAPNPADKCKACNCNLYGTMKQQSSCNP
VTGQCECLPHVTGQDCGACDPGFYNLQSGQGCERCDCHALGSTNGQCDIR
TGQCECQPGITGQHCERCEVNHFGFGPEGCKPCDCHPEGSLSLQCKDDGR
CECREGFVGNRCDQCEENYFYNRSWPGCQECPACYRLVKDKVADHRVKLQ
ELESLIANLGTGDEMVTDQAFEDRLKEAEREVMDLLREAQDVKDVDQNLM
DRLQRVNNTLSSQISRLQNIRNTIEETGNLAEQARAHVENTERLIEIASR
ELEKAKVAAANVSVTQPESTGDPNNMTLLAEEARKLAERHKQEADDIVRV
AKTANDTSTEAYNLLLRTLAGENQTAFEIEELNRKYEQAKNISQDLEKQA
ARVHEEAKRAGDKAVEIYASVAQLSPLDSETLENEANNIKMEAENLEQLI
DQKLKDYEDLREDMRGKELEVKNLLEKGKTEQQTADQLLARADAAKALAE
EAAKKGRDTLQEANDILNNLKDFDRRVNDNKTAAEEALRKIPAINQTITE
ANEKTREAQQALGSAAADATEAKNKAHEAERIASAVQKNATSTKAEAERT
FAEVTDLDNEVNNMLKQLQEAEKELKRKQDDADQDMMMAGMASQAAQEAE
INARKAKNSVTSLLSIINDLLEQLGQLDTVDLNKLNEIEGTLNKAKDEMK
VSDLDRKVSDLENEAKKQEAAIMDYNRDIEEIMKDIRNLEDIRKTLPSGC
FNTPSIEKP

The amino acid sequence for human laminin gamma 2 (LAMC2) (GenBank Accession No. NP_{—}005553.2) is as follows: (SEQ ID NO: 17)

MPALWLGCCLCFSLLLPAARATSRREVCDCNGKSRQCIFDRELHRQTGNG
FRCLNCNDNTDGIHCEKCKNGFYRHRERDRCLPCNCNSKGSLSARCDNSG
RCSCKPGVTGARCDRCLPGFHMLTDAGCTQDQRLLDSKCDCDPAGIAGPC
DAGRCVCKPAVTGERCDRCRSGYYNLDGGNPEGCTQCFCYGHSASCRSSA
EYSVHKITSTFHQDVDGWKAVQRNGSPAKLQWSQRHQDVFSSAQRLDPVY
FVAPAKFLGNQQVSYGQSLSFDYRVDRGGRHPSAHDVILEGAGLRITAPL
MPLGKTLPCGLTKTYTFRLNEHPSNNWSPQLSYFEYRRLLRNLTALRIRA
TYGEYSTGYIDNVTLISARPVSGAPAPWVEQCICPVGYKGQFCQDCASGY
KRDSARLGPFGTCIPCNCQGGGACDPDTGDCYSGDENPDIECADCPIGFY
NDPHDPRSCKPCPCHNGFSCSVMPETEEVVCNNCPPGVTGARCELCADGY
FGDPFGEHGPVRPCQPCQCNNNVDPSASGNCDRLTGRCLKCIHNTAGIYC
DQCKAGYFGDPLAPNPADKCRACNCNPMGSEPVGCRSDGTCVCKPGFGGP
NCEHGAFSCPACYNQVKIQMDQFMQQLQRMEALISKAQGGDGVVPDTELE
GRMQQAEQALQDILRDAQISEGASRSLGLQLAKVRSQENSYQSRLDDLKM
TVERVRALGSQYQNRVRDTHRLITQMQLSLAESEASLGNTNIPASDHYVG
PNGFKSLAQEATRLAESHVESASNMEQLTRETEDYSKQALSLVRKALHEG
VGSGSGSPDGAVVQGLVEKLEKTKSLAQQLTREATQAEIEADRSYQHSLR
LLDSVSRLQGVSDQSFQVEEAKRIKQKADSLSSLVTRHMDEFKRTQKNLG
NWKEEAQQLLQNGKSGREKSDQLLSRANLAKSRAQEALSMGNATFYEVES
ILKNLREFDLQVDNRKAEAEEAMKRLSYISQKVSDASDKTQQAERALGSA
AADAQRAKNGAGEALEISSEIEQEIGSLNLEANVTADGALAMEKGLASLK
SEMREVEGELERKELEFDTNMDAVQMVITEAQKVDTRAKNAGVTIQDTLN
TLDGLLHLMDQPLSVDEEGLVLLEQKLSRAKTQINSQLRPMMSELEERAR
QQRGHLHLLETSIDGILADVKNLENIRDNLPPGCYNTQALEQQ

The amino acid sequence for human laminin gamma 3 (LAMC3) (GenBank Accession No. NP_{—}006050.3) is as follows: (SEQ ID NO: 18)

MAAAALLLGLALLAPRAAGAGMGACYDGAGRPQRCLPVFENAAFGRLAQA
SHTCGSPPEDFCPHVGAAGAGAHCQRCDAADPQRHHNASYLTDFHSQDES
TWWQSPSMAFGVQYPTSVNITLRLGKAYEITYVRLKFHTSRPESFAIYKR
SRADGPWEPYQFYSASCQKTYGRPEGQYLRPGEDERVAFCTSEFSDISPL
SGGNVAFSTLEGRPSAYNFEESPGLQEWVTSTELLISLDRLNTFGDDIFK
DPKVLQSYYYAVSDFSVGGRCKCNGHASECGPDVAGQLACRCQHNTTGTD
CERCLPFFQDRPWARGTAEAAHECLPCNCSGRSEECTFDRELFRSTGHGG
RCHHCRDHTAGPHCERCQENFYHWDPRMPCQPCDCQSAGSLHLQCDDTGT
CACKPTVTGWKCDRCLPGFHSLSEGGCRPCTCNPAGSLDTCDPRSGRCPC
KENVEGNLCDRCRPGTFNLQPHNPAGCSSCFCYGHSKVCASTAQFQVHHI
LSDFHQGAEGWWARSVGGSEHPPQWSPNGVLLSPEDEEELTAPEKFLGDQ
RFSYGQPLILTFRVPPGDSPLPVQLRLEGTGLALSLRHSSLSGPQDAGHP
REVELRFHLQETSEDVAPPLPPFHFQRLLANLTSLRLRVSPGPSPAGPVF
LTEVRLTSARPGLSPPASWVEICSCPTGYTGQFCESCAPGYKREMPQGGP
YASCVPCTCNQHGTCDPNTGICVCSHHTEGPSCERCLPGFYGNPFAGQAD
DCQPCPCPGQSACTTIPESREVVCTHCPPGQRGRRCEVCDDGFFGDPLGL
FGHPQPCHQCQCSGNVDPNAVGNCDPLSGHCLRCLHNTTGDHCEHCQEGF
YGSALAPRPADKCMPCSCHPQGSVSEQMPCDPVTGQCSCLPHVTARDCSR
CYPGFFDLQPGRGCRSCKCHPLGSQEDQCHPKTGQCTCRPGVTGQACDRC
QLGFFGFSIKGCRACRCSPLGAASAQCHENGTCVCRPGFEGYKCDRCHDN
FFLTADGTHCQQCPSCYALVKEEAAKLKARLTLTEGWLQGSDCGSPWGPL
DILLGEAPRGDVYQGHHLLPGAREAFLEQMMSLEGAVKAAREQLQRLNKG
ARCAQAGSQKTCTQLADLEAVLESSEEEILHAAAILASLEIPQEGPSQPT
KWSHLATEARALARSHRDTATKIAATAWRALLASNTSYALLWNLLEGRVA
LETQRDLEDRYQEVQAAQKALRTAVAEVLPEAESVLATVQQVGADTAPYL
ALLASPGALPQKSRAEDLGLKAKALEKTVASWQHMATEAARTLQTAAQAT
LRQTEPLTKLHQEARAALTQASSSVQAATVTVMGARTLLADLEGMKLQFP
RPKDQAALQRKADSVSDRLLADTRKKTKQAERMLGNAAPLSSSAKKKGRE
AEVLAKDSAKLAKALLRERKQAHRRASRLTSQTQATLQQASQQVLASEAR
RQELEEAERVGAGLSEMEQQIRESRISLEKDIETLSELLARLGSLDTHQA
PAQALNETQWALERLRLQLGSPGSLQRKLSLLEQESQQQELQIQGFESDL
AEIRADKQNLEAILHSLPENCASWQ

The RGD-oligopeptides described herein are distinguished by their ability to mimick ECM proteins, such as vitronectin, laminin, and fibronectin, to promote cell adhesion to a particular surface. Assays for detecting the cell adhesive properties of a putative RGD-oligopeptide are known in the art. In one embodiment, a tissue culture plate is coated with the putative RGD-oligopeptide (i.e., embedded in PCL) and a predetermined number of cells are added to the plate. The cells are incubated under normal growing conditions and allowed to adhere for a predetermined amount of time. For example, the predetermined amount of time may be 1 hour, 2 hours, 6 hours, 12 hours, 18 hours, 24 hours, or 48 hours. Then the supernatant is removed and the cells from the supernatant and/or the cells present on the plate are counted by standard counting methods (i.e., hemacytometer or cell counting apparatus). The percentage of cells adhered (or percentage of cells that did not adhere) can be calculated. RGD-oligopeptides suitable for use in the present disclosure will have at least 1%, 2%, 5%, 10%, 15%, 20%, 25%, 30%, 35%, 40%, 45%, 50%, 60%, 70%, 80%, 90% or 100% of the cells attached to the RGD-oligopeptide-coated plate. Alternatively, the RGD-oligopeptides suitable for use in the present disclosure will have at least 1%, 2%, 5%, 10%, 15%, 20%, 25%, 30%, 35%, 40%, 45%, 50%, 60%, 70%, 80%, 90% or 100% increase in the number of cells that adhere to the putative RGD-oligopeptide than to the plate without the RGD-oligopeptide. Other methods and assay for determining cell adhesion to the putative RGD-oligopeptides would be apparent to the ordinarily skilled person in the art.

Described herein is a hybrid matrix suitable for priming surfaces for cell culture, directed cell differentiation, and transplantation. For example, the hybrid matrix is manufactured as a film or as a coating for regular lab-ware. The hybrid matrix comprises PCL with embedded RGD-oligopeptides. The hybrid matrix described herein increases the adhesive properties of the PCL scaffold (e.g., at least 80% of cells attach) and maintains long-term culture (7 days), which is required for appropriate differentiation.

The hybrid matrix is produced by dissolving an RGD-oligopeptide in dichloromethane (or another organic solvent such as chloroform), followed by mixing with a PCL solution. The solution is subsequently applied to a silicon wafer for film manufacturing or to cell-ware for coating.

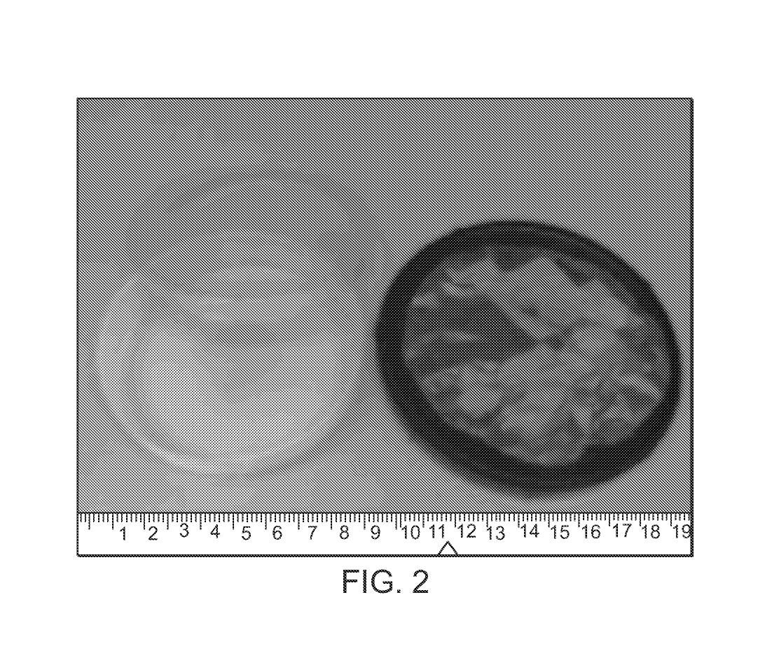

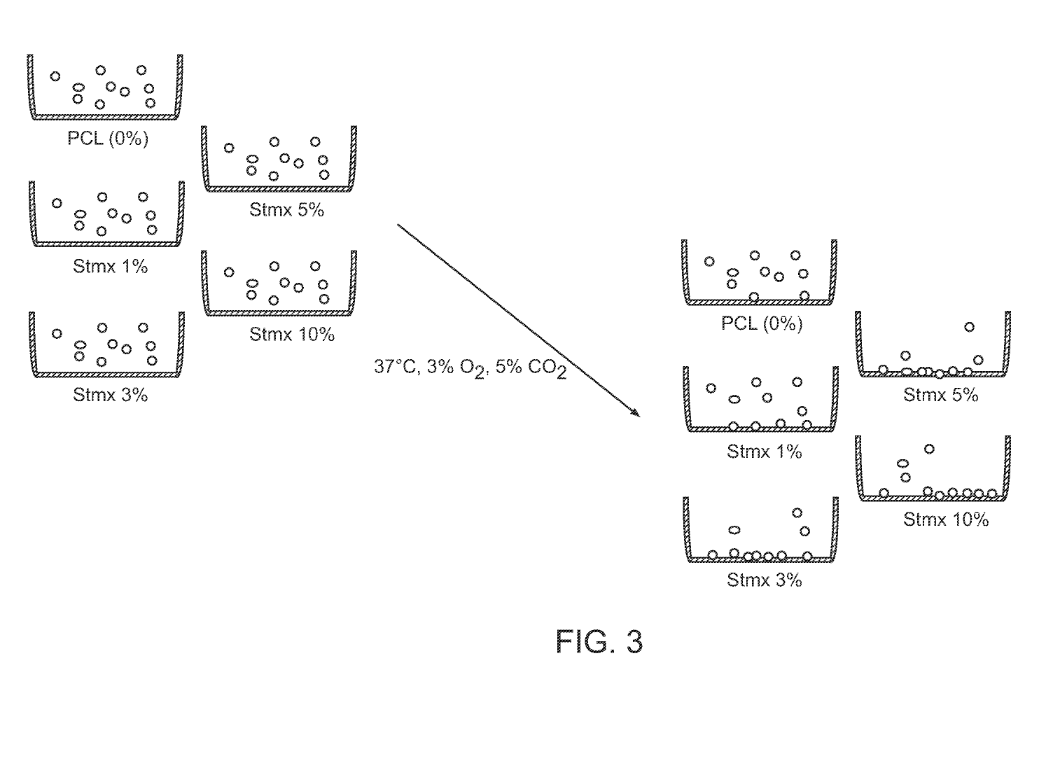

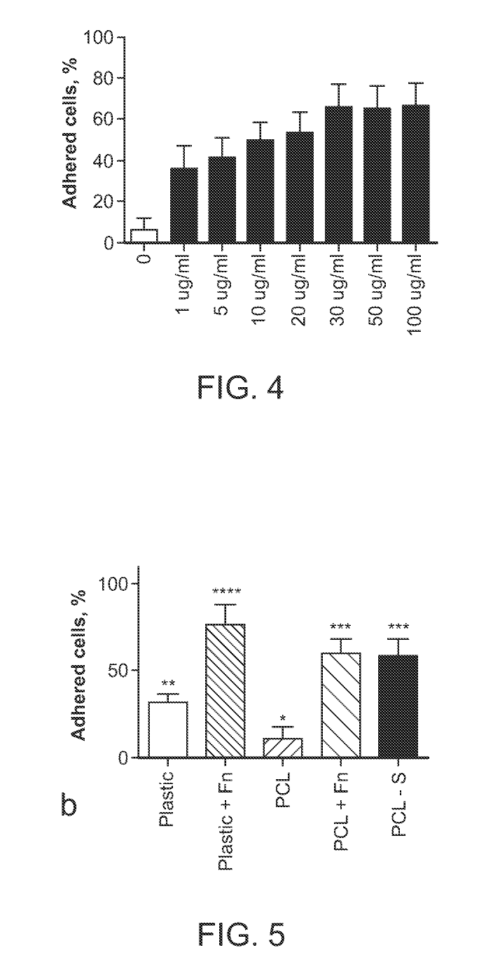

As shown in FIG. 2, RGD-oligopeptides were incorporated in the PCL polymer at different concentrations. The scaffold was also manufactured in several versions: a) as a thin film (thickness may be controlled by changing PCL concentration in the solution); b) as coating of cell culture labware (Petri dishes) of different diameters. In the adhesion experiments, the optimal dose of Synthemax® II-SC(RGD-oligopeptide) in the initial solution was identified as 3% (FIG. 3). As shown in FIG. 4, comparative analysis with other substrates revealed that Synthemax® II-SC incorporation into PCL has equal adhesive properties as fibronectin-coated PCL surface. In the proliferation experiments, Synthemax® II-SC incorporation into the PCL increases the ability of a surface to maintain proliferation (FIG. 5). Differentiation experiments showed that hRPC differentiated to express markers of differentiated photoreceptor cells, such as Nr1, recoverin, and rhodopsin.

Described herein, hRPCs grown on a hybrid scaffold of Synthemax®II-SC and PCL were next transplanted into damaged retinas in a mouse model of retinal degeneration. Specifically, the cells were injected into the subretinal space and localization and functional activity was assessed after 3 week. The results showed that cells grown on the hybrid scaffold were able to successfully integrate into at least the outer nuclear layer of the retina, and electroretinography recording analysis showed that the cells were able to restore electrical responses equally as compared with hRPCs grown on fibronectin-coated PCL surfaces.

The results presented herein demonstrate that that Synthemax® II was successfully incorporated into PCL, and that the hybrid surface was manufactured in different formats, such as films of controlled thickness and a culture flask coating. Additionally, the hybrid surface has adhesive properties equal to conventional fibronectin coating of PCL. Furthermore, incorporation of Synthemax® did not change the inhibitory effect of PCL on hRPC proliferation and promotes differentiation to a mature phenotype of differentiated photoreceptor cells. Cells grown or differentiated on the hybrid matrix described herein were successfully transplanted and integrated into damaged retinal tissue, and were able to restore retinal function.

Thus, all of the data described herein demonstrates that the hybrid matrix described herein is suitable for use for culturing and/or differentiating cells for retinal transplantation.

In all of the following experiments, Synthemax® II-SC substrate (Corning) (vitronectin-mimicking RGD oligopeptide) was used as an example of RGD-containing oligopeptide.

### EXAMPLE 1

### Preparation of Hybrid Matrix

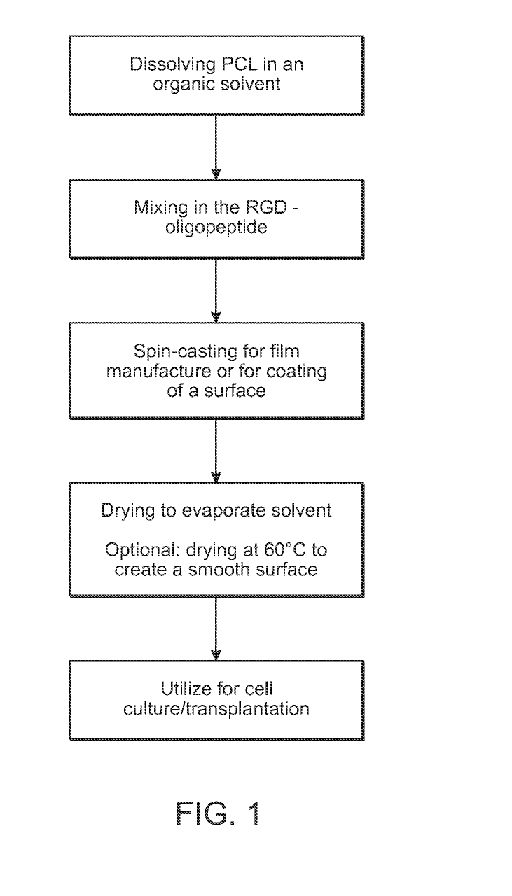

To prepare hybrid scaffolds, 10 mg of Synthemax® powder was dissolved in 95% ethanol, and the required volume of this solution is mixed with 10% PCL solution in dichloromethane. After mixing, PCL-Synthemax® solution is poured onto the surface to be coated and spun at 1500 revolutions per minute (rpm) for 30 seconds to create a thin film coating of the desired thickness. The films or culture dishes are then dried for 1 hour at 45° C. A schematic diagram showing an exemplary method of making a hybrid matrix is provided in FIG. 1. A photograph comparing petri dish coated with either the hybrid matrix (Synthemax® II-SC/PCL) compared to a petri dish coated with thin polymer film is shown in FIG. 2.

### EXAMPLE 2

### Dose-Dependent Adhesion

A dose-dependent experiment was performed to assess the optimal dose of Synthemax® (schematic of the experimental design shown in FIG. 3). Five dilutions of Synthemax® II-SC were initially prepared: 0% (PCL only), 1%, 3%, 5%, and 10%. Additional dilutions of Synthemax® II-SC with PCL were prepared at 0.1%, 0.5% and 2%. Three 35 mm petri dishes (Costar tissue-culture treated and coated with Synthemax® II-SC) were prepared for each dilution. After washing with Hank's Balanced Salt Solution (HBSS) twice, 200,000 hRPC were plated into the individual petri dishes. The plates were then incubated (37° C., 3% O₂, 5% CO₂ and 100% humidity) for 30 minutes. The media was then collected and centrifuged at 1500 rpm at 18° C. for 5 minutes. The supernatant was then removed and the pellet re-suspended in 2 mL of Ultraculture medium (Lonza). Floating cells were counted twice using a hemacytometer and Trypan blue (Sigma-Aldrich). Adhered cells were then harvested from the petri dishes after being incubated (37° C., 5 minutes) with 2 mL of TrypZean (Sigma-Aldrich) per petri dish. They were also counted. The percent of adherent cells was calculated and as shown in FIG. 4. A student T-test revealed no difference in the adhesion for 30, 50, and 100 μg/ml preparations (p=0.57). Moreover, the adhesion in the 30, 50, and 100 μg/ml preparations was higher compared to 10 μg/ml preparations (p<0.0001). Thus, the saturation dose of Synthemax® II-SC was identified as 30 μg/ml, or 3%.

### EXAMPLE 3

### Comparison of Adhesive Properties of Culture Surfaces

An additional experiment was performed to compare the adhesive properties of Synthemax® II-SC to various other surfaces (FIG. 5). The selected surfaces were plastic (Costar tissue-culture treated), fibronectin (200 μg/mL), PCL, fibronectin-coated PCL, and Synthemax®-coated PCL (3% dilution of Synthemax® II-SC). Three 35 mm petri dishes (Costar tissue-treated) were used for each different surface and washed twice with HBSS. Cells were plated at a density of 100,000 cells in each individual petri dish and incubated for 30 minutes (37° C., 3% O₂, 5% CO₂ and 100% humidity). The same process was repeated to count both floating cells and adhered cells as described previously.

### EXAMPLE 4

### Cell Proliferation on Culture Surfaces

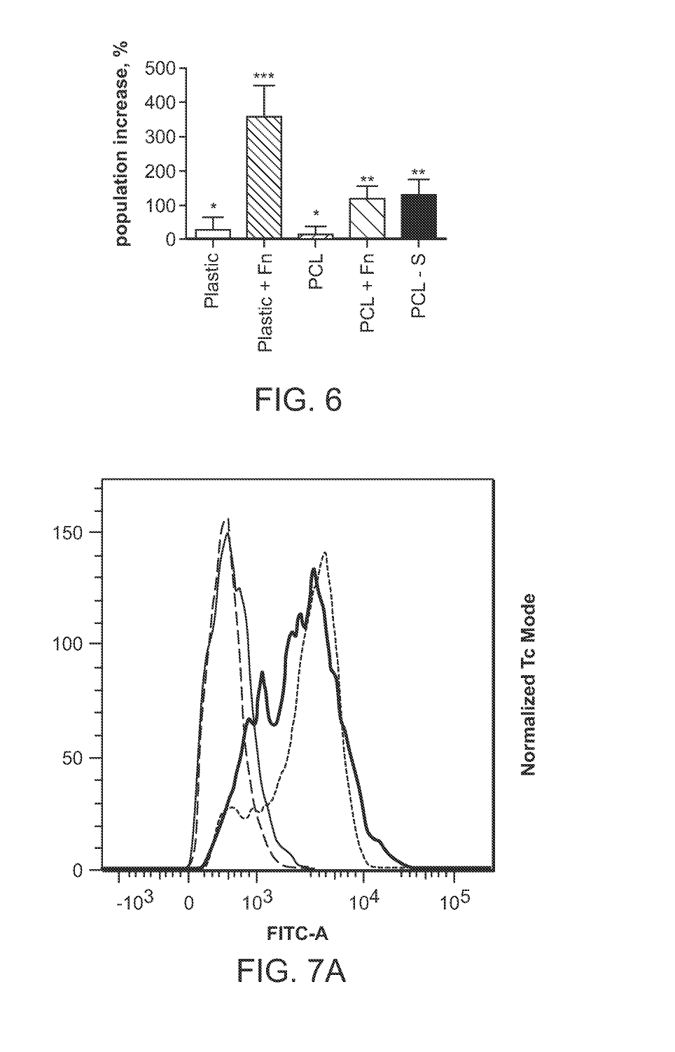

To evaluate the proliferative properties of Synthemax® II-SC/PCL, proliferation on Synthemax® II-SC/PCL was compared to proliferation on different cell culture surfaces (FIG. 6). The selected surfaces were plastic (Costar tissue-culture treated), fibronectin (200 μg/mL), PCL, fibronectin-coated PCL, and Synthemax®-coated PCL (3% dilution of Synthemax® II-SC). Three petri dishes were used for each of the different surfaces. First, human retinal progenitor cells (hRPCs) were plated at a density of 20,000 cells per square centimeter in full media, followed by incubation for 72 hours (37° C., 3% O2, 5% CO₂ and 100% humidity). The media was then removed and the adhered cells harvested with TrypZean (Sigma-Aldrich). They were counted twice per petri dish using Trypan blue (Sigma-Aldrich) and a hemacytometer. The percentage increased proliferation as determined by cell counting was calculated, as shown in FIG. 6. The hybrid scaffolds PCL only, PCL with fibronectin, and PCL with Synthemax® II-SC all inhibited the proliferation of hRPCs. The hybrid matrix comprising PCL with Synthemax® II-SC inhibited the proliferation of the hPRCs in the same manner as fibronectin and PCL-coated plates. Thus, incorporation of Synthemax® II-SC does not change the inhibitory effect of PCL on hRPC proliferation.

### EXAMPLE 5

### Cell Differentiation

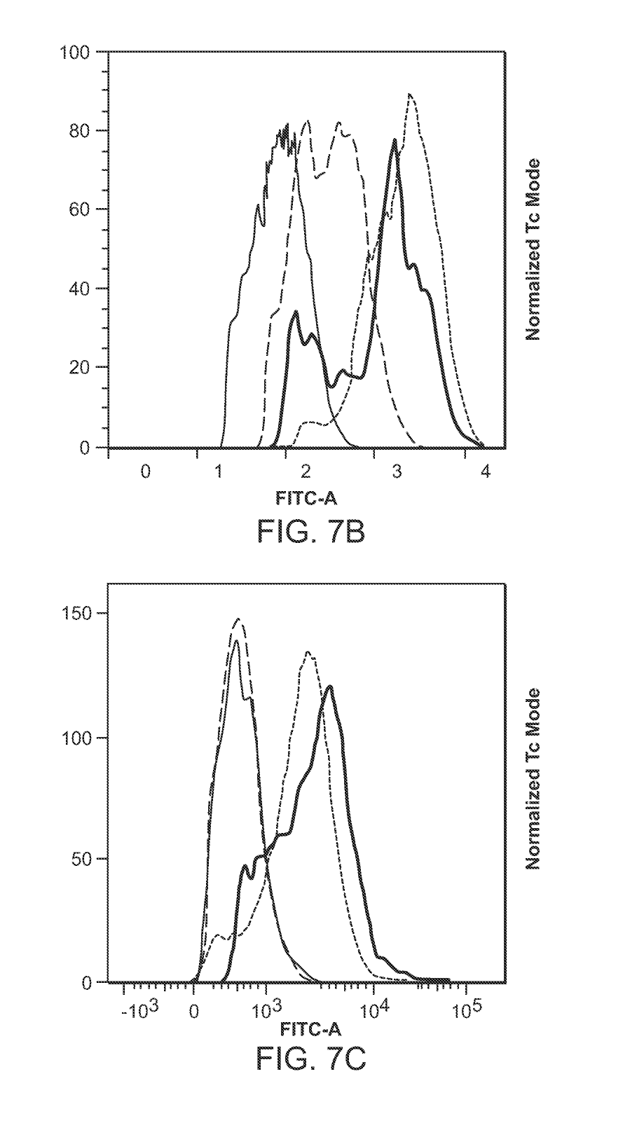

hRPCs were grown on fibronectin and PCL-coated plates or Synthemax® II-SC and PCL-coated plates. Cells were cultured and differentiated using standard methods of differentiation, such as supplementing the media with various differentiation factors, for 7 days. Cells were harvested and stained with antibodies detecting rod-specific marker expression (Nr1, recoverin, rhodopsin) or a control antibody (isotype control). Antibodies suitable for recognizing mature photoreceptor-specific markers are commercially available from companies such as Santa Cruz Biotechnology and Cell Signaling Technologies. Stained cells were then sorted and marker expression was determined by flow cytometry. Histograms of marker expression are shown in FIGS. 7A-C. The expression profiles for the cells grown on different surfaces are shown for the rod-specific marker Nr1 (FIG. 7A), recoverin (FIG. 7B), and rhodopsin (FIG. 7C) before and after 7 days of differentiation. Isotype antibody control is shown in black. As shown in FIGS. 7A-C, the negative isotype control and before differentiation shows no staining for Nr1 and rhodopsin, and minimal recoverin staining. In contrast, differentiation of hRPCs on fibronectin and PCL-coated dishes and Synthemax® II-SC-coated dishes caused increased expression of all rod-specific marker genes, Nr1, recoverin, and rhodopsin, as demonstrated by the shift in the expression curve to the left of each histogram plot. As such, the hybrid scaffold of Synthemax ® II-SC incorporated into PCL causes increased rod-specific marker expression, thereby indicating differentiation of the hRPCs towards mature photoreceptor cells.

### EXAMPLE 6

### In Vivo Characterization of Transplanted Cells Cultured on Hybrid Matrix

The functional and qualitative characteristics of cells cultured on hybrid matrix were assessed in vivo. Rd1 mutant mice are art-recognized models of retinal degeneration. hRPCs were cultured on a variety of hybrid scaffolds, including plastic only (control), fibronectin-coated dishes, fibronectin and PCL-coated dishes, and Synthemax® II-SC-coated dishes. For example, the cells were differentiated on the different surfaces for 7 days. The cells were delivered into the subretinal space of host rd1^{-/-} mice with degenerated retinas. Three weeks after transplantation, the retinas were prepared for electroretinography recording analysis to assess retinal function or for immunofluorescent analysis.

To assess the localization of the cultured cells after transplantation, retinas were stained with nuclei stain, DAPI, and were subsequently examined under a fluorescent microscope to detect GFP signal. The hRPC cells express GFP were shown to integrate into the retina, for example, into the outer nuclear layer. Furthermore, the transplanted cells survived for at least 3 weeks after transplantation.

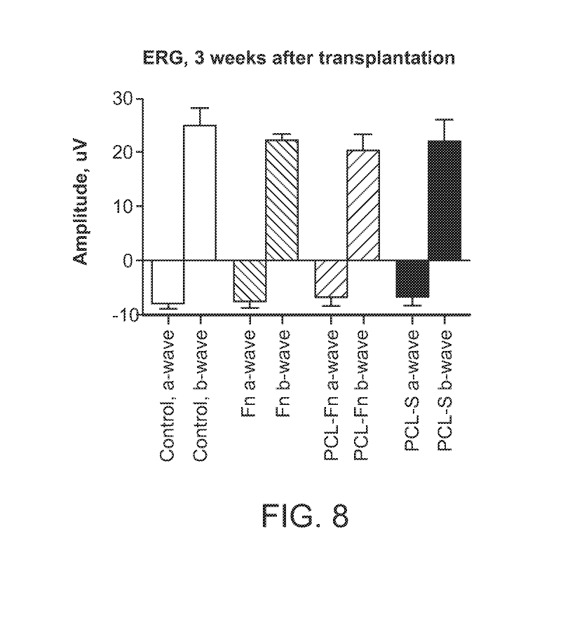

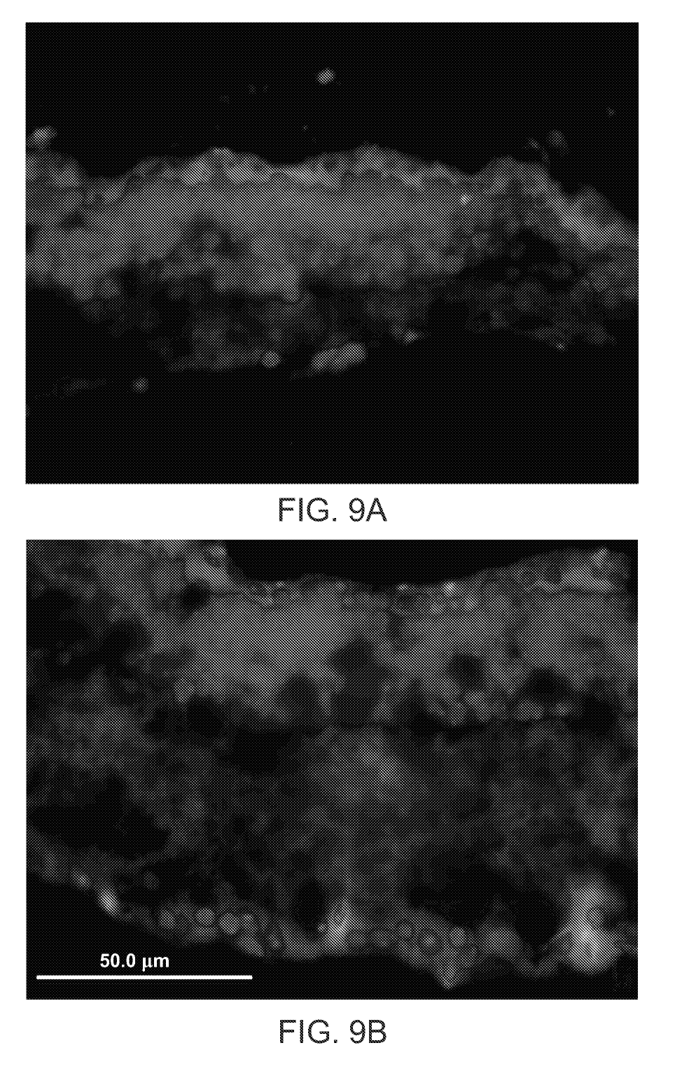

The in vivo functional capabilities of the cells were assessed by electroretinography recording analysis. After photopic stimulation (50 cd*s/m²), a-wave and b-wave amplitudes were measured, and quantitated as shown in FIG. 8. The electroretinography results show that there is no difference in the retinal function of the rd1 hosts between all of the groups. Thus, culturing hRPCs on Synthemax®II-SC and PCL-coated plates produces cultured cells that are capable of restoring retina function in degenerated retinal tissue. These results taken together demonstrate that the hybrid scaffold of Synthemax®II-SC and PCL can be used to differentiate hRPC towards photoreceptor cells that are suitable for transplantation, integration, and treatment of retinal degeneration.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A hybrid matrix comprising poly(caprolactone)(PCL) comprising an RGD-containing oligopeptide (R: arginine; G: glycine; D: aspartic acid) embedded or interspersed in said PCL.

2. The hybrid matrix of claim 1, wherein said RGD-oligopeptide comprises an RGD-containing vitronectin-mimicking oligopeptide, an RGD-containing laminin-mimicking oligopeptide, or an RGD-containing fibronectin-mimicking oligopeptide.

3. The hybrid matrix of claim 2, wherein said RGD-containing vitronectin-mimicking oligopeptide comprises Synthemax® II-SC substrate (SEQ ID NO: 1), or wherein said RGD-containing laminin-mimicking oligopeptide comprises the amino acid sequence set forth in SEQ ID NO: 2, or wherein RGD-containing fibronectin-mimicking oligopeptide comprises the amino acid sequence set forth in SEQ ID NO: 3 or 4.

4. The hybrid matrix of claim 3, wherein said Synthemax® II-SC is between 1% and 25% by weight of said hybrid matrix.

5. The hybrid matrix of claim 1, wherein said hybrid matrix comprises a stiffness about 10 times softer, about 100 times softer, or about 1,000 times softer than tissue culture plastic.

6. The hybrid matrix of claim 1, wherein said hybrid matrix is in the form of a coating.

7. The hybrid matrix of claim 6, wherein said coating is suitable for application to a tissue culture plate.

8. The hybrid matrix of claim 7, wherein said cells adhere to said tissue culture plate.

9. The hybrid matrix of claim 8, wherein 80% of cells adhere to said tissue culture plate.

10. The hybrid matrix of claim 8, wherein said cells adhere to said tissue culture plate for about 7 days.

11. The hybrid matrix of claim 1, wherein said hybrid matrix does not comprise a fibronectin coating.

12. The hybrid matrix of claim 1, wherein said hybrid matrix is in the form of a wafer or thin film.

13. The hybrid matrix of claim 12, wherein said film is smooth.

14. The hybrid matrix of claim 1, wherein said RGD-oligopeptide is covalently bound to said PCL with glutaraldehyde.

15. The hybrid matrix of claim 1, wherein said hybrid matrix is suitable for transplantation.

16. A method of producing a hybrid matrix comprising:
dissolving a PCL solution in an organic solvent;
mixing an RGD-oligopeptide with said PCL solution,
thereby producing said hybrid matrix.

17. The method of claim 16, wherein said organic solvent is dichloromethane or chloroform.

18. A method of culturing one or more cells comprising allowing said cells to grow on a hybrid matrix comprising PCL comprising embedded RGD-oligopeptide.

19. The method of claim 18, wherein said cells are ocular cells.

20. The method of claim 18, wherein said cells are retinal progenitor cells, retinal pigment epithelium cells, photoreceptor precursor cells, neurons, limbal stem cells, transient amplifying corneal epithelial cells, keratinocytes, mesenchymal stem cells, or induced pluripotent stem cells.

21. The method of claim 18, wherein said cells are human cells.

22. The method of claim 18, wherein proliferation of said cells is maintained on said hybrid matrix.

23. The method of claim 18, wherein proliferation of said cells is inhibited on said hybrid matrix.

24. The method of claim 18, wherein said cells differentiate on said hybrid matrix.

25. The method of claim 18, further comprising transplanting said cells into a damaged tissue or organ.

26. The method of claim 25, wherein said damaged tissue comprises the retina or cornea.

27. A method of differentiating cells comprising culturing said cells on a hybrid matrix comprising PCL comprising an RGD-containing oligopeptide, wherein said RGD-containing oligopeptide is interspersed or embedded in said PCL, thereby producing differentiated cells.

28. The method of claim 27, wherein said cells are grown on said hybrid matrix for 7 days.

29. The method of claim 27, wherein said cells are retinal progenitor cells, retinal pigment epithelium cells, photoreceptor precursor cells, neurons, limbal stem cells, transient amplifying corneal epithelial cells, keratinocytes, mesenchymal stem cells, or induced pluripotent stem cells.

30. The method of claim 27, wherein said cells are human cells.

31. The method of claim 27, wherein said differentiated cells express one or more marker of a mature cell.

32. The method of claim 31, wherein said one or more marker comprises Nr1, recoverin, and rhodopsin.

33. The method of claim 27, wherein said differentiated cells are mature photoreceptor cells.

34. The differentiated cells produced using the method of claim 27.
